(19) United States
(12) Patent Application Publication
(10) Pub. No.: US 2026/0144935 A1
(43) Pub. Date: May 28, 2026

(54) DRIVE ASSEMBLY FOR A DRUG DELIVERY DEVICE AND RELATED METHODS

(71) Applicant: AMGEN INC., Thousand Oaks, CA (US)

(72) Inventor: Jan Mikael JENSEN, Herlev (DK)

(73) Assignee: AMGEN INC., Thousand Oaks, CA (US)

(21) Appl. No.: 19/400,327

(22) Filed: Nov. 25, 2025

Related U.S. Application Data
(60) Provisional application No. 63/726,048, filed on Nov. 27, 2024.

(51) Int. Cl. A61M 5/20; A61M 5/315

(52) U.S. Cl. CPC A61M 5/2033; A61M 5/31501; A61M 5/31578; A61M 2005/2073; A61M 2005/31508; A61M 2202/0007; A61M 2202/04; A61M 2205/8281; A61M 2207/10

(57) ABSTRACT

Drive assemblies for a drug delivery device and related methods are disclosed. A drive assembly comprises a casing having a proximal end, a distal end, and a longitudinal axis extending between the proximal end and the distal end, a plunger rod guide operably coupled to the casing, a spring at least partially disposed within the casing and wound around at least a portion of the plunger rod guide, and a lock component selectively coupled with the casing and configured to receive at least a portion of the plunger rod guide therein. The spring is configured to exert a force on the plunger rod guide that causes the plunger rod guide to rotate. The lock component is configured to prevent rotational movement of the plunger rod guide relative to the casing when the lock component is coupled with the casing.

### CROSS-REFERENCE TO RELATED APPLICATIONS

Priority is claimed to U.S. Provisional Patent Application No. 63/726,048, filed Nov. 27, 2024.

### FIELD OF DISCLOSURE

The present disclosure relates to drive assemblies for drug delivery devices, and, more particularly, drive assemblies for devices for automatically injecting a drug into a patient.

### BACKGROUND

A general aversion to exposed needles, as well as health and safety issues, have led to the development of drug delivery devices, such as injectors and autoinjectors, which conceal a needle or other insertion member prior to use and which automate or semi-automate various aspects of an injection process. Such devices offer a variety of benefits as compared with traditional forms of drug delivery including, for example, delivery via a conventional syringe.

A drug delivery device may incorporate various mechanisms to implement various automated or semi-automated features. Such features may include automatically covering a needle in a pre-delivery and/or post-delivery state, automatically activating a drive mechanism, automatically indicating to the user that drug delivery is complete, among other features. Certain such features are activated by the application of an external force, for example, by a user.

However, such features may increase the number of components that need to be manufactured and assembled to form the complete drug delivery device and may complicate the manufacturing and assembly processes. For example, the automated drive mechanism alone may comprise numerous components that need to be manufactured and manually assembled into a drug delivery device. As such, the time required to assemble a single drug delivery device, let alone batches of drug delivery devices, may significantly increase.

The present disclosure sets forth drive assemblies for drug delivery devices and related methods to address one or one of the needs and challenges mentioned herein and other related needs and challenges.

### SUMMARY

One aspect of the present disclosure provides a drive assembly for a drug delivery device. The drive assembly comprises a casing having a proximal end, a distal end, and a longitudinal axis extending between the proximal end and the distal end, a plunger rod guide operably coupled to the casing, a spring at least partially disposed within the casing and wound around at least a portion of the plunger rod guide in an energized state, and a lock component selectively coupled with the casing and configured to receive at least a portion of the plunger rod guide therein. The spring is configured to exert a force on the plunger rod guide that causes the plunger rod guide to rotate. The lock component is configured to prevent rotational movement of the plunger rod guide relative to the casing when the lock component is coupled with the casing to lock the spring in the energized state.

In some embodiments, the casing may include a bottom surface, a plurality of sidewalls extending from the bottom surface and defining a cylindrical body portion, and a lid. The bottom surface, the plurality of sidewalls, and the lid may be integrally formed. In some embodiments, the bottom surface, the plurality of sidewalls, and the lid of the casing may be stamped from a single sheet of metal. In some embodiments, the casing may have a flat configuration, where the bottom surface, the plurality of sidewalls, and the lid are substantially flat, and an assembled configuration, where the bottom surface, the plurality of sidewalls, and the lid are folded to form the cylindrical body portion of the casing. In some embodiments, the casing may further include at least one opening formed through the lid, and at least one sidewall of the plurality of sidewalls may include a protrusion extending from the at least one sidewall. When the casing is in the assembled configuration, the protrusion may be configured to clinch through the at least one opening on the lid to lock the lid in a closed configuration.

In some embodiment, the casing may further include a cavity formed through the bottom surface and the lid, and at least a portion of the plunger rod guide may be disposed through the cavity. In some embodiments, the lid may include one or more cut-out portions, and the lock component may be configured to selectively engage the one or more cut-out portions to lock the spring in the energized state. In some embodiments, the lock component may have a proximal end, a distal end, and at least one hook adjacent to the distal end, and the at least one hook may be configured to selectively engage the one or more cut-out portions of the lid to prevent rotational movement of the plunger rod guide relative to the casing. In some embodiments, the lid may include a plurality of cut-out portions, and the lock component may have a plurality of hooks adjacent to the distal end thereof. Each of the hooks may be configured to selectively engage a respective one of the plurality of cut-out portions.

In some embodiments, the plunger rod guide may be configured to receive a rod portion of a plunger therein and guide rotational movement of the plunger. In some embodiments, the spring may include a torsion spring and may be formed from a single metal band. The metal band may have a first end and a second end. The first end of the metal band may include a first hook, and the second end of the metal band may include a second hook. The first hook may be configured to engage at least a portion of the plunger rod guide to lock the first end of the metal band onto the plunger rod guide. The second hook may be configured to engage a sidewall of the casing. In some embodiments, the plunger rod guide, the spring, and the lock component may be axially aligned with the longitudinal axis. In some embodiments, the drive assembly may be configured to be assembled within a drug delivery device via an axial assembly, and the lock component may be configured to be removed from the casing once the drive assembly is assembled within the drug delivery device.

Another aspect of the present disclosure provides a drive assembly for a drug delivery device. The drive assembly comprises a casing having a proximal end, a distal end, and a longitudinal axis extending between the proximal end and the distal end, a plunger rod guide operably coupled to the casing, and a spring at least partially disposed within the casing and wound around at least a portion of the plunger rod guide in an energized state. The spring is configured to exert a force on the plunger rod guide that causes the plunger rod guide to rotate.

In some embodiments, the casing may include a bottom surface, a plurality of sidewalls extending from the bottom surface and defining a cylindrical body portion, and a lid. The bottom surface, the plurality of sidewalls, and the lid may be integrally formed. In some embodiments, the bottom surface, the plurality of sidewalls, and the lid of the casing may be stamped from a single sheet of metal. The casing may have a flat configuration, where the bottom surface, the plurality of sidewalls, and the lid are substantially flat, and an assembled configuration, where the bottom surface, the plurality of sidewalls, and the lid are folded to form the cylindrical body portion of the casing. In some embodiments, the casing may further include at least one opening formed through the lid, and at least one sidewall of the plurality of sidewalls may include a protrusion extending from the at least one sidewall. When the casing is in the assembled configuration, the protrusion may be configured to clinch through the opening to lock the lid in a closed configuration.

In some embodiments, the casing may further include a cavity formed through the bottom surface and the lid, and wherein at least a portion of the plunger rod guide is disposed through the cavity. In some embodiments, the plunger rod guide may be configured to receive a rod portion of a plunger therein and guide rotational movement of the plunger. In some embodiments, the spring may include a torsion spring and may be formed from a single metal band. The metal band may have a first end and a second end. The first end of the metal band may include a first hook, and the second end of the metal band may include a second hook. The first hook may be configured to engage at least a portion of the plunger rod guide to lock the first end of the metal band onto the plunger rod guide, and the second hook may be configured to engage a sidewall of the casing. In some embodiments, the plunger rod guide and the spring may be axially aligned with the longitudinal axis. In some embodiments, the drive assembly may be configured to be assembled within a drug delivery device via an axial assembly.

Another aspect of the present disclosure provides a lock component for a drug delivery device. The lock component comprises a hollow body portion having a proximal end, a distal end, and a longitudinal axis extending between the proximal end and the distal end, and a plurality of hooks positioned adjacent to the distal end of the hollow body portion. The hollow body portion includes a distal body portion extending proximally along the longitudinal axis from the distal end thereof and a proximal body portion extending proximally along the longitudinal axis from the distal body portion. The distal body portion and the proximal body portion are shaped to accommodate at least a portion of a plunger rod guide therein.

In some embodiments, the distal body portion may have a substantially circular cross-section perpendicular to the longitudinal axis, and the proximal body portion may have a non-circular cross-section perpendicular to the longitudinal axis. In some embodiments, the plurality of hooks may be positioned along a circumference of the distal end of the hollow body portion. In some embodiments, the plurality of hooks may be configured to selectively engage a spring casing of a drive assembly for a drug delivery device during axial assembly of the drive assembly within the drug delivery device. When the plurality of hooks are selectively engaged with the spring casing, the lock component may be configured to prevent rotational movement of the plunger rod guide relative to the casing and lock a spring disposed within the spring casing in an energized state. In some embodiments, the plurality of hooks may be configured to disengage from the spring casing after axial assembly of the drive assembly within the drug delivery device.

Yet another aspect of the present disclosure provides a method of assembling a drive assembly for a drug delivery device. The method comprises providing a casing having a bottom surface, a plurality of sidewalls extending from the bottom surface and defining a cylindrical body portion, and a lid, inserting at least a portion of a plunger rod guide through a cavity formed through the bottom surface and the lid of the casing, feeding a metal band into the casing through an opening in the plurality of sidewalls, winding the metal band around a portion of the plunger rod guide within the casing to form a spring, and selectively coupling a lock component with the lid of the casing. The lock component is configured to prevent rotational movement of the plunger rod guide relative to the casing and lock the spring in an energized state when the lock component is coupled with the lid.

In some embodiments, the bottom surface, the plurality of sidewalls, and the lid of the casing are integrally formed. In some embodiments, the bottom surface, the plurality of sidewalls, and the lid of the casing are stamped from a single sheet of metal. In some embodiments, the lid may include one or more cut-out portions, and the lock component may include at least one hook adjacent to a distal end thereof. Selectively coupling the lock component with the lid of the casing may further comprise selectively engaging the at least one hook with the one or more cut-out portions to lock the spring in the energized state.

In some embodiments, the lock component may be configured to receive at least a proximal portion of the plunger rod guide when the lock component is coupled to the lid of the casing to prevent rotational movement of the plunger rod guide relative to the casing. In some embodiments, the plunger rod guide may be configured to receive a rod portion of a plunger therein and guide rotational movement of the plunger. In some embodiments, the spring may be a torsion spring, and the metal band may include a first hook at a first end thereof and a second hook at a second end thereof. The first hook may be configured to engage at least a portion of the plunger rod guide to lock the first end of the metal band onto the plunger rod guide, and the second hook may be configured to engage a sidewall of the casing. In some embodiments, the method may further comprise axially assembling the drive assembly into a drug delivery device while the lock component is coupled to the lid of the casing, and removing the lock component from the casing.

Another aspect of the present disclosure provides a method of manufacturing a drive assembly for a drug delivery device. The method comprises stamping, via a stamping tool, a sheet of metal to form a bottom surface, a plurality of sidewalls, and a lid of a casing, cutting, via a cutting tool, the bottom surface, the plurality of sidewalls, and the lid from the sheet of metal, folding the bottom surface, the plurality of sidewalls, and the lid to form the casing, inserting at least a portion of a plunger rod guide through a cavity formed through the bottom surface and the lid of the casing, feeding a metal band into the casing through an opening in the plurality of sidewalls of the casing, winding the metal band around a portion of the plunger rod guide within the casing to form a spring, and selectively coupling a lock component with the lid of the casing. The bottom surface, the plurality of sidewalls, and the lid are integrally formed. Additionally, the lock component is configured to prevent rotational movement of the plunger rod guide relative to the casing and lock the spring in an energized state when the lock component is coupled with the lid.

In some embodiments, the casing may further include at least one opening formed through the lid, and at least one sidewall of the plurality of sidewalls may include a protrusion extending from the at least one sidewall. The method may further comprise clinching the protrusion through the at least one opening on the lid to lock the lid in a closed configuration. In some embodiments, the method may further comprise forming a first hook at a first end of the metal band and forming a second hook at a second end of the metal band. The method may further comprise coupling the first hook to at least a portion of the plunger rod guide to lock the first end of the metal band onto the plunger rod guide, and coupling the second hook to a sidewall of the casing.

In some embodiments, the lock component may be configured to receive at least a proximal portion of the plunger rod guide when the lock component is coupled to the lid of the casing to prevent rotational movement of the plunger rod guide relative to the casing. In some embodiments, the lid may include one or more cut-out portions, and the lock component may include at least one hook adjacent to a distal end thereof. Selectively coupling the lock component to the lid of the casing may further comprise selectively engaging the at least one hook with the one or more cut-out portions on the lid. In some embodiments, the plunger rod guide may be configured to receive a rod portion of a plunger therein and guide rotational movement of the plunger. In some embodiments, the spring may be a torsion spring configured to exert a force on the plunger rod guide that causes the plunger rod guide to rotate.

Another aspect of the present disclosure provides a drug delivery device comprising a housing having a proximal end, a distal end, and a longitudinal axis extending between the proximal end and the distal end thereof, a container including a needle and filled or fillable with a drug, the container being disposed within the housing, and a drive assembly disposed within the housing and activatable to drive a plunger in a distal direction to expel the drug from the container through the needle. The drive assembly comprises a casing, a plunger rod guide operably coupled to the casing, a spring at least partially disposed within the casing and wound around at least a portion of the plunger rod guide in an energized state, and a lock component selectively coupled with the casing and configured to receive at least a portion of the plunger rod guide therein. The spring is configured to exert a force on the plunger rod guide that causes the plunger rod guide to rotate. Additionally, the lock component is configured to prevent rotational movement of the plunger rod guide relative to the casing when the lock component is coupled with the casing to lock the spring in the energized state.

In some embodiments, the casing may include a bottom surface, a plurality of sidewalls extending from the bottom surface and defining a cylindrical body portion, and a lid. The bottom surface, the plurality of sidewalls, and the lid may be integrally formed. The casing may further include at least one opening formed through the lid, and at least one sidewall of the plurality of sidewalls may include a protrusion extending from the at least one sidewall. The protrusion may be configured to clinch through the at least one opening on the lid to lock the lid in a closed configuration. In some embodiments, the casing may further include a cavity formed through the bottom surface and the lid, and at least a portion of the plunger rod guide may be disposed through the cavity. The lid may include one or more cut-out portions, and the lock component may be configured to selectively engage the one or more cut-out portions to lock the spring in the energized state. In some embodiments, the lock component may have a proximal end, a distal end, and at least one hook adjacent to the distal end, and the at least one hook may be configured to selectively engage the one or more cut-out portions of the lid to prevent rotational movement of the plunger rod guide relative to the casing. In some embodiments, the lid may include a plurality of cut-out portions, and the lock component may have a plurality of hooks adjacent to the distal end thereof. Each of the hooks may be configured to selectively engage a respective one of the plurality of cut-out portions.

In some embodiments, the spring may include a torsion spring and may be formed from a single metal band having a first end and a second end. The first end of the metal band may include a first hook and the second end of the metal band may include a second hook, and the first hook may be configured to engage at least a portion of the plunger rod guide to lock the first end of the metal band onto the plunger rod guide. The second hook may be configured to engage a sidewall of the casing. In some embodiments, the container, the plunger, the plunger rod guide, the spring, and the lock component may be axially aligned with the longitudinal axis of the housing. In some embodiments, the drive assembly may be configured to be assembled within a drug delivery device via an axial assembly, and the lock component may be configured to be removed from the casing once the drive assembly is assembled within the drug delivery device.

In some embodiments, upon activation of the drive assembly, the plunger rod guide may be configured to receive a force output by the spring and rotate relative to the housing. The plunger may be threadingly engaged with the plunger rod guide such that a rotational movement of the plunger rod guide causes the plunger to rotate and translate linearly in the distal direction to expel the drug from the container. In some embodiments, the plunger may be at least partially disposed within the plunger rod guide

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 is a cross-sectional view of an exemplary drug delivery device in a pre-injection state, in accordance with various embodiments of the present disclosure.

FIG. 2A is a perspective view of an exemplary drive assembly, in accordance with various embodiments of the present disclosure.

FIG. 2B is a cross-sectional side view of the drive assembly of FIG. 2A, in accordance with various embodiments of the present disclosure.

FIG. 2C is a top view of the drive assembly of FIG. 2A, in accordance with various embodiments of the present disclosure.

FIG. 3A is a perspective view of the drive assembly of FIG. 2A without a lock component, in accordance with various embodiments of the present disclosure.

FIG. 3B is a cross-sectional side view of the drive assembly of FIG. 3A, in accordance with various embodiments of the present disclosure.

FIG. 3C is a cross-sectional perspective view of the drive assembly of FIG. 3A, in accordance with various embodiments of the present disclosure.

FIG. 4A is a perspective view of an exemplary lock component, in accordance with various embodiments of the present disclosure.

FIG. 4B is a partial perspective view of the lock component of FIG. 4A, in accordance with various embodiments of the present disclosure.

FIG. 5 is a partial perspective view of the lock component of FIG. 4A engaged with a casing, in accordance with various embodiments of the present disclosure.

FIG. 6 is a perspective view of an exemplary plunger rod guide, in accordance with various embodiments of the present disclosure.

FIG. 7A is a perspective view of an exemplary casing, in accordance with various embodiments of the present disclosure.

FIG. 7B is a side view of the casing of FIG. 7A, in accordance with various embodiments of the present disclosure.

FIG. 7C is a top view of the casing of FIG. 7A, in accordance with various embodiments of the present disclosure.

FIG. 7D is a bottom view of the casing of FIG. 7A, in accordance with various embodiments of the present disclosure.

FIG. 8A is a perspective view of an exemplary spring band, in accordance with various embodiments of the present disclosure.

FIG. 8B is a cross-sectional perspective view of the spring band of FIG. 8A wound around the plunger rod guide of FIG. 6 within the casing of FIG. 7A, in accordance with various embodiments of the present disclosure.

FIG. 8C is a top view of the spring band of FIG. 8A wound around the plunger rod guide of FIG. 6 within the casing of FIG. 7A, in accordance with various embodiments of the present disclosure.

FIG. 8D is a perspective view of the spring band of FIG. 8A disposed within the casing of FIG. 7A, in accordance with various embodiments of the present disclosure.

FIG. 9 is a flowchart illustrating an exemplary method of assembling the drive assembly of FIG. 2A, in accordance with various embodiments of the present disclosure.

FIG. 10 is a flowchart illustrating an exemplary method of manufacturing and assembling the drive assembly of FIG. 2A, in accordance with various embodiments of the present disclosure.

FIG. 11 is a partial perspective view of another embodiment of the drive assembly of FIG. 2A, in accordance with various embodiments of the present disclosure.

FIG. 12 illustrates a step of inserting the plunger rod guide of FIG. 6 in the casing of FIG. 7A as a part of the method of assembling the drive assembly shown in FIG. 9, in accordance with various embodiments of the present disclosure.

FIG. 13 illustrates a cross-sectional side view of the plunger rod guide of FIG. 6 inserted in the spring casing of FIG. 7A, in accordance with various embodiments of the present disclosure.

FIG. 14 illustrates a step of feeding the spring band of FIG. 8A onto the plunger rod guide of FIG. 6 in the casing of FIG. 7A as a part of the method of assembling the drive assembly shown in FIG. 9, in accordance with various embodiments of the present disclosure.

FIG. 15 illustrates a cross-sectional perspective view and cross-sectional top views of the spring band of FIG. 8A fed onto the plunger rod guide of FIG. 6, in accordance with various embodiments of the present disclosure.

FIG. 16 illustrates a step of winding the spring band of FIG. 8A around the plunger rod guide of FIG. 6 in the casing of FIG. 7A as a part of the method of assembling the drive assembly shown in FIG. 9, in accordance with various embodiments of the present disclosure.

FIG. 17 illustrates a perspective view of the spring band of FIG. 8A wound around the plunger rod guide of FIG. 6, in accordance with various embodiments of the present disclosure.

FIG. 18 illustrates a cross-sectional top view of the spring band of FIG. 8A wound around the plunger rod guide of FIG. 6, in accordance with various embodiments of the present disclosure.

FIG. 19 illustrates a step of inserting the lock component of FIG. 4A onto the casing of FIG. 7A as a part of the method of assembling the drive assembly shown in FIG. 9, in accordance with various embodiments of the present disclosure.

FIG. 20 illustrates a partial perspective view of the lock component of FIG. 4A inserted onto the casing of FIG. 7A, in accordance with various embodiments of the present disclosure.

FIG. 21 illustrates another partial perspective view of the lock component of FIG. 4A inserted onto the casing of FIG. 7A, in accordance with various embodiments of the present disclosure.

FIG. 22 illustrates another partial perspective view of the lock component of FIG. 4A inserted onto the casing of FIG. 7A, in accordance with various embodiments of the present disclosure.

FIG. 23 illustrates a partial side view of the lock component of FIG. 4A inserted onto the casing of FIG. 7A, in accordance with various embodiments of the present disclosure.

### DETAILED DESCRIPTION

The present disclosure generally relates to the configuration and operation of a drug delivery device. While most embodiments are described in the context of a hand-held injector such as autoinjector, the scope of the present disclosure in not limited to such injectors. Other drug delivery devices for which the present disclosure is applicable include, without limitation, wearable injectors such as on-body injectors which adhere (e.g., via an adhesive) to a patient's skin, ambulatory pumps worn on a patient's clothing, and hybrid autoinjector/on-body injectors.

In general, the drive assembly and the drug delivery device of the present disclosure are designed to limit the number of parts or components that need to be assembled during the final assembly process. Moreover, the drive assembly is designed to minimize or eliminate the need for manual manufacturing and assembly of parts of components of the drug delivery device. The drive assembly of the present disclosure is pre-assembled and loaded into a unitary structure prior to a final assembly stage. Accordingly, during the final assembly stage, multiple components that make up the drive assembly can be assembled into the drug delivery device in a single step. Additionally, the drive assembly can be fully tested to, for example, ensure accurate performance of the spring and ensure the input and output torque of the spring, for example, are within a specified range. Moreover, the pre-assembly process and/or the final assembly process may be automated to increase efficiency. This simplifies the final assembly stage and potentially allows it to be performed at a facility that is less costly or demanding to maintain than a facility where the earlier manufacturing steps are performed. Additionally, the automated pre-assembly and/or final assembly process significantly increases efficiency, manufacturability, accuracy, and quality. Other benefits and advantages are also possible and will be apparent to one of ordinary skill in the art reviewing the present disclosure.

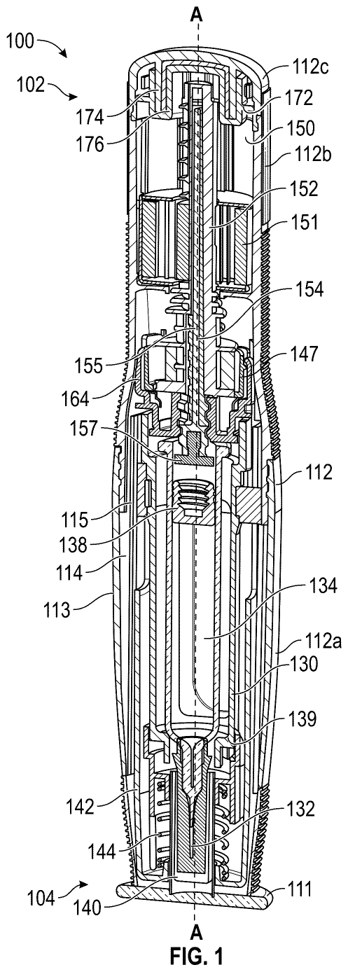

Referring now to FIGS. 1-3B, illustrated are several cross-sectional views of an embodiment of a drug delivery device 100 for injecting a drug, which may also be referred to herein as a medicament or drug product. The drug may be, but is not limited to, various biologicals such as peptides, peptibodies, or antibodies. The drug may be in a fluid or liquid form, although the disclosure is not limited to a particular state. In certain liquid formulations, the drug may have a relatively high viscosity.

Various implementations and configurations of the drug delivery device 100 are possible. The drug delivery device 100 may be configured as a single-use, disposable injector, or alternatively, a multiple-use reusable injector. The drug delivery device 100 may be configured as an autoinjector for self-administration by a patient, although the drug delivery device 100 can also be used by a caregiver or a formally trained healthcare provider (e.g., a doctor or nurse) to administer an injection. In embodiments where the drug delivery device 100 is configured as an autoinjector or pen-type injector, the drug delivery device 100 may be held in the hand of a patient or user over the duration of drug delivery, also referred to herein as dosing. In alternative embodiments, where drug delivery may be delayed or take several minutes or hours to complete, the drug delivery device 100 may be configured as an on-body injector which is releasably attached to the patient's skin via, for example, an adhesive disposed at or applied to an exterior surface of the drug delivery device 100.

The configuration of various components included in the drug delivery device 100 may depend on the operational state of the drug delivery device 100. The drug delivery device 100 may have a pre-injection or storage state, an injection state, and a post-injection state, although fewer or more states are also possible. The pre-injection state may correspond to the configuration of the drug delivery device 100 following assembly and prior to activation by a patient or user (e.g., activation of an energy source of the drug delivery device 100). The pre-injection state may exist in the time between when the drug delivery device 100 leaves a manufacturing facility and when a patient or user activates the drug delivery device 100 for drug delivery. The injection state may correspond to the configuration of the drug delivery device 100 over the course of drug delivery. In certain embodiments, the injection state may also exist in the time between activation and drug delivery, during which a needle or other delivery member is inserted into the patient. The post-injection state may correspond to the configuration of the drug delivery device 100 after drug delivery is complete and/or when a stopper is arranged in an end-of-dose position within a container.

FIG. 1 illustrates the drug delivery device 100 in a pre-injection state. The drug delivery device 100 includes a proximal end 102 and a distal end 104, with the distal end 104 being defined by a cap 111 in the pre-injection state. The drug delivery device 100 also includes a longitudinal axis extending between the proximal end 102 and the distal end 104. The cap 111 may be removably attached to an outer casing or housing 112. Prior to use, a patient or user may detach the cap 111 from the outer housing 112 to expose an opening providing access to a subcutaneous delivery member such as a needle or cannula. In the illustrated embodiment, the outer housing 112 is constructed in three separate (i.e., discrete) and rigidly connected components, arranged from the distal end 104 to proximal end 102 as follows: a front housing portion 112a, a rear housing portion 112b, and a rearmost housing portion 112c. By way of their rigid connection, the housing portions 112a, 112b, and 112c may be prevented from moving relative to each other. In some versions, the housing portions 112a, 112b, and 112c may be made of different materials and/or thickness depending on the amount of load, if any, that they bear in the pre-injection, injection, and/or post-injection state. In alternative embodiments, the housing portions 112a, 112b, and 112c may be integrally formed with each other such that the housing portions 112a, 112b, and 112c form a single, unitary structure.

The outer housing 112 may include an exterior surface 113 configured to be held by a patient or user over the course of an injection, and an interior surface 114 defining a hollow interior space 115 in which various moving components are disposed. The outer housing 112 may generally have an elongate shape such as a hollow cylinder or tube, or any other suitable shape.

The drug delivery device 100 may also include a container 130 that is filled (e.g., pre-filled) or fillable (e.g., filled by a patient or user at the time of use of the drug delivery device 100) with a drug. The container 130 is disposed or disposable within the interior space 115 of the outer housing 112, particularly the front housing portion 112a. The container 130 may be pre-loaded within the interior space 115 by a manufacturer, or loaded within the interior space 115 by a patient or user at the time of use. In the present embodiment, the container 130 is configured as pre-filled syringe having a staked and rigid hollow needle 132 protruding from its distal end. The needle 132 is connected in fluid communication with an internal bore or reservoir 134 of the container 130. In alternative embodiments, the container 130 may be configured as a cartridge that is not initially in fluid communication with the needle 132 or other subcutaneous delivery member. Instead, the subcutaneous delivery member and/or a fluid pathway connection assembly may be actuated upon activation of the drug delivery device 100 in order to establish fluid communication between the reservoir 134 and the subcutaneous delivery member. In addition to or as an alternative to the needle 132, in some embodiment a flexible cannula may be included. Whereas the needle 132 may be constructed of a rigid material such as metal, the flexible cannula may be constructed of a relatively soft material such as plastic.

The needle 132 may include a hollow interior passageway extending between its proximal and distal ends to allow the drug to flow through the needle 132 during an injection. An opening, or multiple openings, may be formed in the distal end of the needle 132 to permit the drug to be delivered subcutaneously to the patient. Furthermore, the distal end of the needle 132 may include a tapered region where the width of the subcutaneous delivery member gradually decreases to a tip. The tip may be sharp enough to pierce at least through the patient's skin and subcutaneous tissue. In some versions, the tip may be sharp enough to penetrate through other tissue as well, including, for example, muscle, arterial walls, and/or bone. A removable shield 140 (e.g., a rigid needle shield) may be removably attached to the container 130 and enclose the distal end of the needle 132, including its tip, to maintain the distal end of the needle 132 in a sterile state prior to use. The removable shield 140 may be connected to the cap 111 such that removal of the cap 111 results in detachment of the removable shield 140 from the container 130, thereby exposing the distal end of the needle 132.

Still referring to FIG. 1, the container 130 may include a rigid wall defining the internal bore or reservoir 134. The wall may be made of glass or plastic. In addition to containing the drug, the reservoir 134 may contain a stopper 138. The stopper 138 may be movably disposed within the container 130 such that it can move in a distal direction along a longitudinal axis of the container 130 from a proximal end of the reservoir 134 to a distal end of the reservoir 134. The stopper 138 may slidably and sealingly contact an interior surface of the wall such that the drug is prevented or inhibited from leaking past the stopper 138 while the stopper 138 is in motion. As a consequence, the stopper 138 is able to expel the drug contained in the reservoir 134 through the needle 132 as the stopper 138 is driven in the distal direction. The proximal end of the container 130 may be open to allow a plunger 154 to extend into the container 130 and push the stopper 138 in the distal direction. The stopper 138 may continue to move in the distal direction until it contacts a proximally-facing portion of the interior surface of the wall. This position of the stopper 138 may be referred to as the end-of-dose position and may correspond to when delivery of the drug to the patient is complete. The stopper 138 may be constructed of rubber or any other suitable material.

As illustrated in FIG. 1, the container 130 may be received, partially or fully, within a carrier or container holder 139. The container holder 139 generally serves as a mount for the container 130, and may be rigidly connected to the container 130 such that the container 130 and the container holder 139 do not move relative to each other during operation of the drug delivery device 100. Additionally, in certain embodiments, the container holder 139 may be rigidly mounted to the front housing portion 112a, such that the container holder 139 and the front housing portion 112a also do not move relative to each other during operation of the drug delivery device 100. In alternative embodiments, the container 130 and/or the container holder 139 may move relative to the front housing portion 112a during operation of the drug delivery device 100. The container holder 139 may have a hollow and generally cylindrical or tubular shape, and it may have various openings or cut-outs formed in its circumferential wall.

With continued reference to FIG. 1, the drug delivery device 100 may also include a guard mechanism for preventing the user or patient from inadvertent contact with the tip of the needle 132 prior to use. The guard mechanism may include a guard member 142 movably disposed at the distal end 104 of the outer housing 112. The guard member 142 may have a hollow and generally cylindrical or tubular shape. The guard mechanism may further include a guard biasing member 144 (e.g., a spring) that urges the guard member 142 towards an extended position protruding from the outer housing 112 when the drug delivery device 100 is not in use (as depicted in FIG. 1), and allows the guard member 142 to retract in the proximal direction into the interior space 115 of the outer housing 112 when the distal end 104 of the drug delivery device 100 is pressed against an injection site. As a consequence of this retraction, the distal end of the needle 132 may be exposed and allowed to penetrate into the injection site. Retraction of the guard member 142 may also have the effect of actuating of a trigger assembly. As depicted in FIG. 1, the trigger assembly includes a trigger ring 147. Retraction of the guard member 142 may push the trigger ring 147 in the proximal direction.

The drug delivery device 100 may further include a drive assembly 150 that stores actuation energy and releases the actuation energy upon activation by a patient or user to drive the stopper 138 through the container 130 for expelling the drug and/or to drive the needle 132 into the patient's tissue. The drive assembly 150 may be disposed within the external housing 112, with at least part of the drive assembly 150 being disposed within the rear housing portion 112b. The drive assembly 150 may include, without limitation, an energy source 151, a plunger rod guide 152, a plunger 154, and a nut 164.

As illustrated in FIG. 1, the plunger 154 includes a rod 155 having a threaded exterior surface, and a washer 157 connected to a distal end of the rod 155. The washer 157 may be rigidly connected to and in direct contact with the rod 155 such that the rod 155 and the washer 157 move together jointly during operation. In alternative embodiments, the rod 155 and the washer 157 may not be discrete components but rather integrally formed with each together as a single, unitary, structure. In the pre-injection state, the washer 157 may be spaced apart from the stopper 138 by a distance, as depicted in FIG. 1. In the injection state, the washer 157 may impact and exert a force or otherwise act on the stopper 138 to drive the stopper 138 through the reservoir 134 in the distal direction.

The energy source 151 may be configured to store mechanical, electrical, and/or chemical energy. Upon activation, the energy source 151 may release or otherwise output this energy in order to generate the motive force needed for actuating one or more components of the drug delivery device 100. The energy source 151 may be a spring (e.g., a torsion spring, such as a helical torsion spring or a spiral torsion spring, a helical compression spring, a helical extension spring, etc.). In the embodiment illustrated in FIG. 1, the energy source 151 is defined by a spiral torsion spring, also sometimes referred to as a watch spring. In the pre-injection state, the spiral torsion spring may be retained in a wound or energized state. Activation of the drug delivery device 100 may release the spiral torsion spring so that it can unwind to a de- or less-energized state. The energy source 151 may output a biasing force in the pre-injection state and the injection state, and also optionally in the post-injection state. The biasing force output by the spiral torsion spring is a rotational force (i.e., a torque).

In some embodiments, the force output by the energy source 151 may vary in magnitude over the course of the pre-injection, injection, and/or post-injection states. In embodiments where the energy source 151 includes a spring, the force output by the energy source 151 may have a largest magnitude in the pre-injection state, then gradually decrease in magnitude over the course of the injection state, and have a smallest magnitude in the post-injection state. Also, in spring embodiments, the magnitude of the force output by the energy source 151 may be constant or un-changing in the pre-injection state and/or the post-injection state. In alternative embodiments, the force output by the energy source 151 may have a constant magnitude throughout the pre-injection, injection, and/or post-injection states.

The plunger rod guide 152 may have a hollow and generally cylindrical or tubular shape, and receive a proximal end of the rod 155 of the plunger 154, in at least the pre-injection state. In some embodiments, an inner diameter of the plunger rod guide 152 may be larger than an outer diameter of the plunger 154 such that the plunger rod guide 152 and the plunger 154 do not contact each other; whereas, in other embodiments, the two components may be in contact or allowed to contact each other. The nut 164 may be ring-shaped, and may receive at least a portion of the distal end of the rod 155 of the plunger 154 in the pre-injection state. The nut 164 may have a threaded interior surface that threadably engages the threaded exterior surface of the rod 155 of the plunger 154. By way of this threaded engagement, rotation of the nut 164 relative to the rod 155 may drive the rod 155 linearly in the distal direction.

The plunger rod guide 152 may be directly connected to the energy source 151 such that the plunger rod guide 152 directly receives, and can be actuated by, the force output by the energy source 151. In the present embodiment, actuation of the plunger rod guide 152 by the energy source 151 involves the plunger rod guide 152 rotating about the plunger 154 in the injection state. A distal end of the plunger rod guide 152 may be rigidly connected to the nut 164 such that the plunger rod guide 152 and the nut 164 rotate together jointly in the injection state. Rotational motion of the nut 164 is converted into distal linear motion of the plunger 154 via the threaded engagement between these two components. The distal linear motion of the plunger 154 is transmitted to the stopper 138, which in turn expels the drug from the container 130 via the needle 132.

In the pre-injection state, the trigger ring 147 may lockingly engage an exterior surface of the plunger rod guide 152, thereby inhibiting or preventing the plunger rod guide 152 from rotating. In the injection state, the trigger ring 147 may be displaced to a position where it no longer engages the plunger rod guide 152, thereby allowing the plunger rod guide 152 to rotate and drive, via the nut 164, the plunger 154 in the distal linear direction.

Still referring to FIG. 1, the drug delivery device 100 may in certain embodiments include a damper assembly 172. The damper assembly 172 generally functions as a shock absorber operable to absorb or dampen a shock impulse caused by the plunger 154 striking the stopper 138. This impulse, if not moderated, may shatter or break the container 130, which may be made of glass, and potentially break other components of the drug delivery device 100 as well. When initially released, the output force of the energy source 151 may be at its greatest magnitude as compared to later in the plunger stroke. As a consequence, the energy source 151 may accelerate the plunger 154 to a relatively high velocity prior to the plunger 154 acting on the stopper 138. The damper assembly 172 may be operable to reduce the velocity of the plunger 154 prior to acting on the stopper 138. As illustrated in FIG. 1, the damper assembly 172 may include a damper housing 174 and a damper member 176. The damper member 176 may be fixedly connected to the plunger rod guide 152 such that two components rotate together jointly in the injection state. In some embodiments, a damping fluid may be sealed within the damper housing 174 and in contact with the damper member 176. The damping fluid may at least initially act to resist rotational motion of the damper member 176, thereby providing the intended damping effect. In alternative embodiments, the damping fluid may be omitted, and instead the damping effect may be achieved via frictional contact between the exterior surface of the damper member 176 and the interior surface of the damper housing 174.

The damper housing 174 may be disposed within and rigidly connected to and in direct contact with the outer housing 112, particularly the rearmost housing portion 112c. In alternative embodiments, the damper housing 174 may be positioned outside of the outer housing 112 and rigidly connected thereto. In such alternative embodiments, the damper housing 174 may correspond to the rearmost housing portion 112c, and thus form a portion of the exterior surface of the drug delivery device 100. The damper housing 174 may possess a hollow and generally cylindrical or tubular shape, and may receive, in full or in part, the damper member 176 such that the damper housing 174 surrounds or partially surrounds the damper member 176. While the damper member 176 may be able to rotate relative to the damper housing 174, other degrees of freedom of the damper member 176 may be restricted.

In some embodiments, the damper member 176 may be ring-shaped and receive a portion of the proximal end of the plunger rod guide 152, as illustrated in FIG. 1. An interior surface of the damper member 176 may directly and slidably contact the exterior surface of the plunger rod guide 152. Friction between these two surfaces during the injection state may provide the desired damping effect. Other embodiments may additionally or alternatively rely on a shock absorbing material and/or hydraulic fluid to provide the desired damping effect.

While the present disclosure describes and/or illustrates certain components of the drug delivery device 100 as being separate, discrete elements, it should be understood that in some embodiments such components may be integrally formed with each other as a single, unitary structure, including components described and/or illustrated as being in direct contact with and/or rigidly connected to each other, so long as such components are not required to move relative to each other during use or operation of the drug delivery device 100.

As discussed above, the drug delivery device 100 incorporates various mechanisms to implement various automated or semi-automated features. For example, the drug delivery device 100 includes a drive assembly 150 that stores actuation energy and, upon activation by a patient or user by retracting the guard member 142, the drive assembly 150 releases the actuation energy to drive the stopper 138 through the container 130 for expelling the drug and/or to drive the needle 132 into the injection site. Such features that are incorporated into the drug delivery device 100 to implement automated or semi-automated features may increase the number of components that need to be not only manufactured but also assembled to form the complete drug delivery device 100. For example, conventional assembly processes may require manually and individually assembling each component of the drive assembly 150, including the energy source 151, the plunger rod guide 152, the plunger 154, and the nut 164, into the drug delivery device 100. Thus, such features may complicate the manufacturing and assembly processes, particularly when the components require manual manufacturing and assembly into the drug delivery device 100. As such, the time required to assemble a single drug delivery device, such as the drug delivery device 100, let alone batches of drug delivery devices, may significantly increase.

The present disclosure addresses one or one of these needs and challenges. For example, in some embodiments, some or all components of the drive assembly 150 may be pre-assembled into a unitary structure prior to assembling the drug delivery device 100. Accordingly, during final assembly of the drug delivery device 100, multiple components of the drive assembly 150 can be assembled into the drug delivery device 100 in a single step. The pre-assembly process and/or the final assembly process may be automated to increase efficiency. Moreover, the present disclosure sets forth an automated system and method for manufacturing the drive assembly 150 to further increase efficiency and manufacturability of the drive assembly 150.

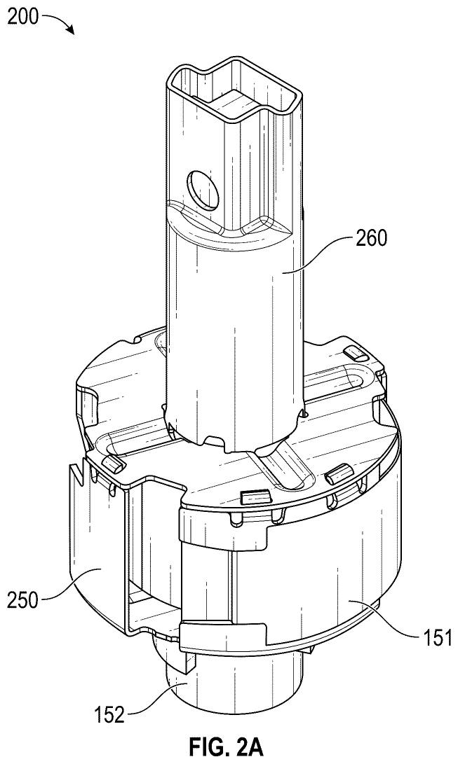

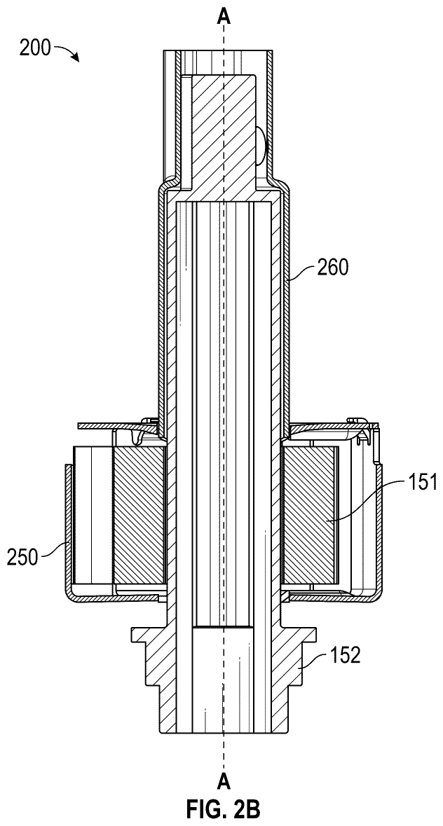

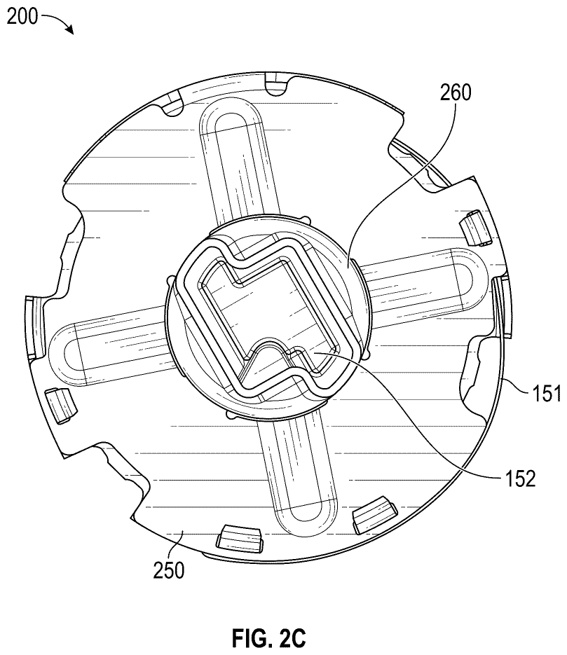

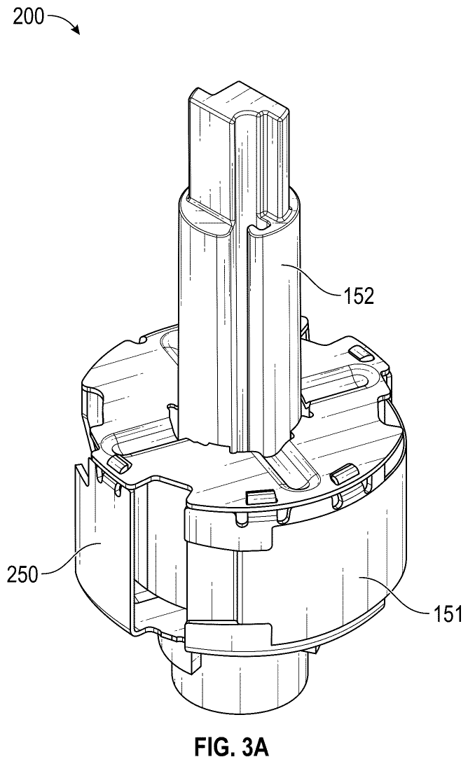

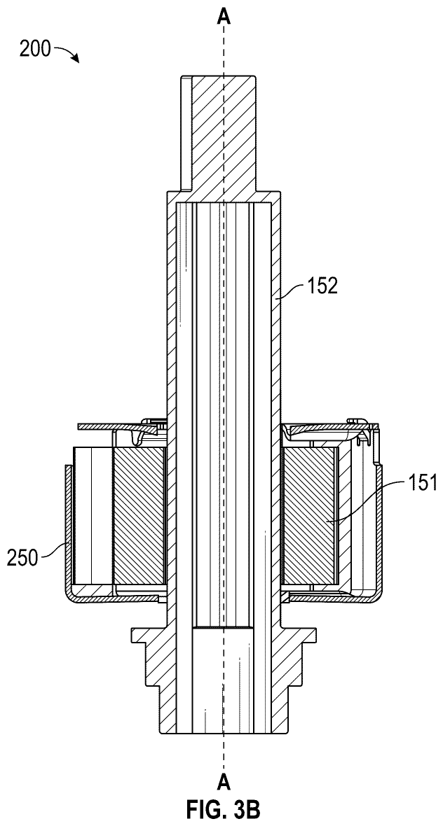

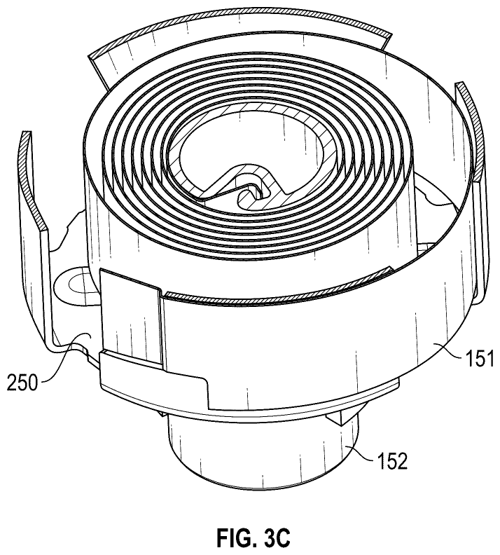

Referring to FIGS. 2A-2C, a pre-assembled drive assembly 200 is shown according to various embodiments of the present disclosure. The drive assembly 200 may illustrate a portion of the drive assembly 150 in FIG. 1 that is pre-assembled into a unitary structure. The drive assembly 200 comprises a casing 250, the plunger rod guide 152 operably coupled to the casing 250, the energy source 151 disposed at least partially within the casing 250, and a lock component 260 removably and/or selectively coupled with the casing 250. The casing 250, the plunger rod guide 152, the energy source 151, and the lock component 260 may be rigidly connected to each other and pre-assembled into a unitary structure to form the drive assembly 200. When pre-assembled, the drive assembly 200 may be fully loaded, and the energy source 151 may be locked in an energized state by the lock component 260. During the final assembly process to form the drug delivery device 100, the drive assembly 200 may be axially assembled and inserted into the drug delivery device 100 in a single step, and the lock component 200 may be removed. FIGS. 3A-3C illustrate the drive assembly 200 when the lock component 200 is removed. Accordingly, the final assembly process in accordance with the embodiments of the present disclosure can not only be automated but also simplified by removing numerous steps that would be required in conventional assembly processes. Additionally, as discussed in more detail below, until the lock component 200 is removed, the lock component 200 may be configured to prevent rotational movement of the plunger rod guide 152 relative to the casing 250 and, thus, lock the energy source 151 in the energized state. Accordingly, the amount of energy stored in the energy source 151 (e.g., output force of the energy source 151) can be controlled, thereby preventing any risk of inconsistent performance of the drive assembly 200. In some embodiments, the drive assembly 200 can be removed from the drug delivery device 100 for inspection and/or for re-use in the drug delivery device 100 and/or a new drug delivery device. Accordingly, at least the components that make up the drive assembly 200 may be re-used, thereby increasing sustainability and reducing manufacturing and material costs.

In some embodiments, one or more components of the drive assembly 200 may be engraved with a barcode identifier to allow traceability of the one or more components of the drive assembly 200. For example, one or more components of the drive assembly 200, such as the casing 250, the plunger rod guide 152, the energy source 151, and/or the lock component 260 may be engraved with a two-dimensional barcode identifier. The barcode identifier can be scanned, for example using an external camera or an external reader, to access data regarding the one or more components of the drive assembly 200. Data regarding the one or more components of the drive assembly 200 may include, for example, the component number, the manufacturer information, the manufacturing date and time, the manufacturing location, the dimensions of the component, or any suitable information related to each component. All data regarding the components of the drive assembly 200 may be stored in batch record and can be easily looked up by scanning the barcode identifier on each component.

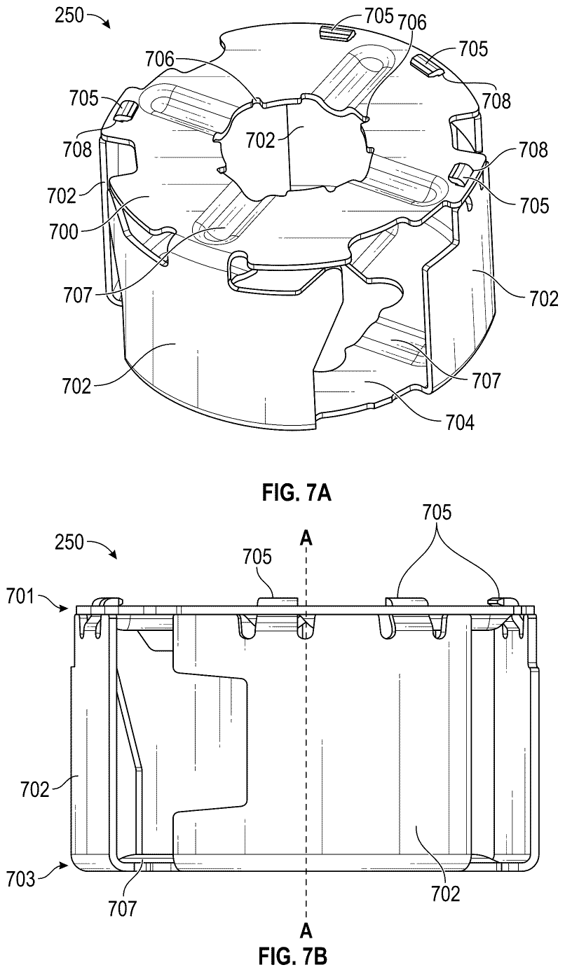

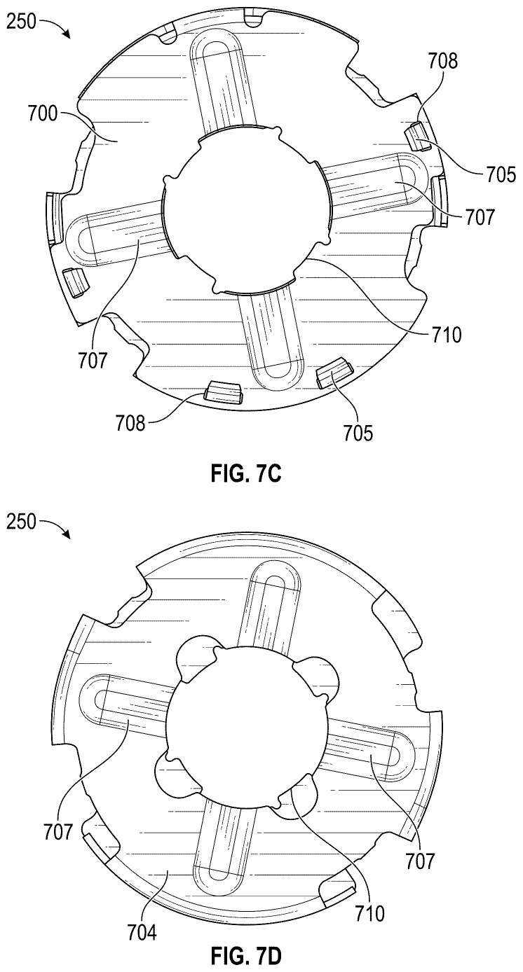

Various components of the drive assembly 200 will now be described in detail with reference to FIGS. 2A-2C, 3A-3C, 4A-4B, 5, 6, 7A-7D, and 8A-8D. As discussed above, the drive assembly 200 comprises the casing 250 shown in FIGS. 7A-7D. In some embodiments, the casing 250 may be made of steel. The casing 250 has a proximal end 701 (FIG. 7B), a distal end 703 (FIG. 7B), and a longitudinal axis A (FIG. 7B) extending between the proximal end 702 and the distal end 703. The casing 250 also has a bottom surface 704, a plurality of sidewalls 702 extending from the bottom surface 704, and a lid 700. In some embodiments, the bottom surface 704, the sidewalls 702, and the lid 700 are integrally formed. For example, the bottom surface 704, the sidewalls 702, and the lid 700 may be formed from a single sheet of material, such as metal. As discussed in more detail below, a metal band or sheet may be inserted on a reel and fed through a stamping tool. The stamping tool may stamp the metal band or sheet, and the stamped metal sheet may be cut using a cutting tool to form the bottom surface 704, the sidewalls 702, and the lid 700. When the stamped metal sheet is cut to form the bottom surface 704, the sidewalls 702, and the lid 700, the casing 250 may be in its flat configuration, where the bottom surface 704, the sidewalls 702, and the lid 700 are substantially flat. Afterwards, the metal sheet may be folded to form the casing 250 having a cylindrical shape. When the metal sheet is folded to form the casing 250 having a cylindrical shape, the casing 250 may be in its assembled configuration, where the bottom surface 704, the sidewalls 702, and the lid 700 are folded to form the cylindrical body portion of the casing 250. For example, the sidewalls 702 may be folded up to define the cylindrical body portion of the casing 250, and the lid 700 may be folded over the sidewalls 702 to form an overlid. FIGS. 7A-7D illustrate the casing 250 in its assembled configuration.

In some embodiments, the casing 250 may have at least one opening 708 formed through the lid 700. In some embodiments, the casing 250 may have a plurality of openings 708 formed through the lid 700. While FIGS. 7A-7D illustrate the casing 250 having four openings 708 formed through the lid 700, the casing 250 may have any number of openings 708, such as one, two, three, five, six, seven, or eight openings 708. One or more sidewalls 702 of the casing 250 may have a protrusion 705 extending from the sidewall 702. For example, as shown in FIGS. 7A-7D, some of the sidewalls 702 may each have one or more protrusions 705 extending from the respective sidewall 702. Although FIGS. 7A-7D illustrate the casing 250 having three sidewalls 702 having one or two protrusions 705 extending from the respective sidewall 702, the casing 250 may have any number of protrusions extending from any number of sidewalls 702. The protrusions 705 may be configured to clinch through a respective opening 708 on the lid 700. For example, when the casing 250 is in its assembled configuration, the sidewalls 702 may be folded up to define the cylindrical body portion of the casing 250. Afterwards, the lid 700 may be folded over the sidewalls 702 to form an overlid. In the assembled configuration, the protrusions 705 extending from the sidewalls 702 may be inserted through the respective openings 708 on the lid 700 and clinched to lock the lid 700 of the casing 250 in a closed configuration. This approach of locking the lid 700 in the closed configuration may be advantageous because it eliminates the need to laser weld the lid 700 onto the casing 250, which may result in inconsistent low pull force of the lid.

In some embodiments, the casing 250 may further include one or more embossments 707 formed on the bottom surface 704 and/or the lid 700. When the energy source 151 is disposed within the casing 250, the embossment(s) 707 may be configured to contact the top and/or bottom of the energy source 151 to control the axial position of the energy source 151. Accordingly, by controlling the axial position of the energy source 151, the embossment(s) 707 may be configured to reduce or minimize friction on the energy source 151 when the energy source 151 unwinds into a less- or de-energized state upon activation of the drive assembly. Additionally, the embossment(s) 707 may limit contact between the energy source 151 and the casing 250 and reduce any friction between the energy source 151 and the casing 250.

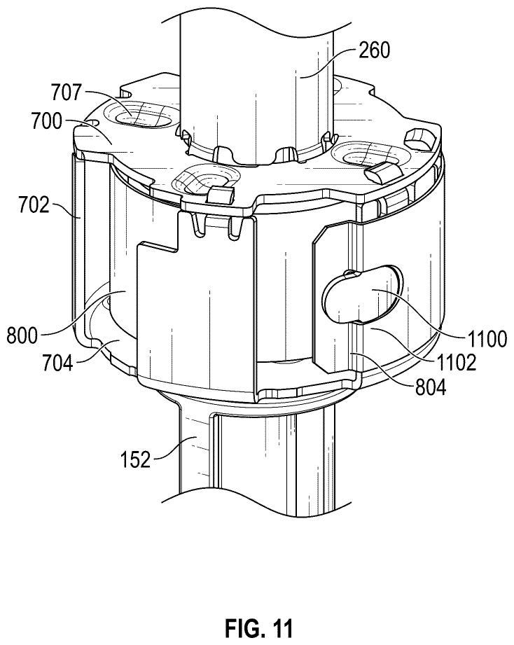

In other embodiments, the one or more embossments may be substantially elliptical in shape. For example, as shown in FIG. 11, the embossment(s) 707 formed on the bottom surface 704 and/or the lid 700 may be substantially elliptically-shaped and may be configured to contact the top and/or bottom of the energy source 151 to control the axial position of the energy source 151 so as to reduce or minimize friction on the energy source 151 when the energy source 151 unwinds into a less- or de-energized state upon activation of the drive assembly.

In some embodiments, the casing 250 may include a cavity 710 (FIGS. 7C and 7D) formed through the bottom surface 704 and the lid 700. The cavity 710 may be axially aligned with the longitudinal axis A of the casing 250. In some embodiments, the cavity 710 may be shaped such that at least a portion of the plunger rod guide 152 may be disposed through the cavity 710. For example, as shown in FIGS. 2A-2C and 3A-3C, the plunger rod guide 152 may be axially inserted through the cavity 710 on the bottom surface 704 and through the cavity 710 on the lid 700 such that at least a portion of the plunger rod guide 152 is disposed within the casing 250. In some embodiments, the cavity 710 may have a diameter in the range between 8 mm and 10 mm. For example, the cavity 710 may have a diameter in the range between 9 mm and 10 mm, or between 9.1 mm and 9.6 mm. The lid 700 of the casing 250 may also include one or more cut-out portions 706. The cut-out portions 706 may be positioned along a perimeter of the cavity 710 on the lid 700. For example, as shown in FIG. 7A, the cut-out portions 706 may be positioned around a perimeter of the cavity 710 on the lid 700. The cut-out portions 706 may be configured to removably and/or selectively engage the lock component 260 to prevent rotational movement of the plunger rod guide 152 relative to the casing 250 and lock the energy source 151 in an energized state.

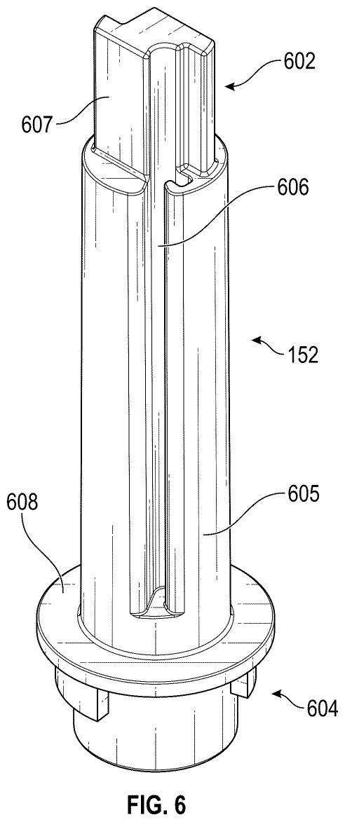

As discussed above, the plunger rod guide 152 may be at least partially disposed within the casing 250 of the drive assembly 200. Referring now to FIG. 6, the plunger rod guide 152 may have a proximal end 602 and a distal end 604. When the plunger rod guide 152 is at least partially disposed within the casing 250, the plunger rod guide 152 may be axially aligned with the longitudinal axis A, as shown in FIGS. 2B and 3B. In some embodiments, the plunger rod guide 152 may be made of plastic. In other embodiments, the plunger rod guide 152 may be made of a metal, such as steel. The plunger rod guide 152 may have a hollow and generally cylindrical or tubular body 605. The plunger rod guide 152 may be configured to receive a portion of the plunger 154 therein. For example, the plunger rod guide 152 may be configured to receive a proximal end of the rod 155 of the plunger 154 therein, in at least the pre-injection state. In some embodiments, an inner diameter of the plunger rod guide 152 may be larger than an outer diameter of the plunger 154 such that the plunger rod guide 152 and the plunger 154 do not contact each other; whereas, in other embodiments, the two components may be in contact or allowed to contact each other. The plunger rod guide 152 may be configured to guide rotational movement of the plunger 154. For example, upon activation of the drive assembly, the plunger rod guide 152 may receive the force output by the energy source 151 and begin rotating in the injection state. The rod 155 of the plunger 154 may be threadingly engaged with the plunger rod guide 152 such that the rotational movement of the plunger rod guide 152 may cause the plunger 154 to rotate. The plunger 154 may translate linearly in a distal direction while rotating, and the distal linear motion of the plunger 154 is transmitted to the stopper 138, which in turn expels the drug from the container 130 via the needle 132.

The plunger rod guide 152 may also have a flange 608 extending radially outward from the body 605 towards the distal end 604 of the plunger rod guide 152. The flange 608 may be configured to limit the axial movement of the plunger rod guide 152 relative to the casing 250. For example, the plunger rod guide 152 may be axially inserted through the cavity 710 on the bottom surface 704 of the casing 250 and through the cavity 710 on the lid 700 of the casing 250 such that at least a portion of the body 605 of the plunger rod guide 152 is disposed within the casing 250. The flange 608 may limit the axial movement of plunger rod guide 152 relative to the casing 250 such that the body 605 of the plunger rod guide 152 cannot be inserted into the casing 250 past the flange 608. The plunger rod guide 152 may further include a proximal portion 607 extending proximally from the body 605 along a longitudinal axis. While the body 605 may be hollow and generally tubular, the proximal portion 607 may not be hollow. In addition, the proximal portion 607 may have a non-circular cross-section that is perpendicular to the longitudinal axis. For example, as shown in FIG. 6, the proximal portion 607 may have a cross-section that is shaped like a “Z” (i.e., Z-shaped). As discussed in more detail below, the proximal portion 607 having a Z-shaped cross-section may be shaped to fit within a corresponding portion of the lock component 260.

In some embodiments, the body 605 of the plunger rod guide 152 may include a recess 606 extending along the longitudinal axis. In some embodiments, the recess 606 may extend along at least a portion of the length of the body 605. In other embodiments, the recess 606 may extend along the entire length of the body 605. The recess 606 may be configured to engage one end of a metal band that is wound to form the energy source 151. During the winding process (described in more detail below), a first end of a metal band may engage the recess 606 to securely lock the metal band onto the plunger rod guide 152. Accordingly, the recess 606 may be configured to prevent disengagement of the metal band during the winding process to form the energy source 151, even if the winding is performed at a significant speed.

Although not shown in FIG. 6, in some embodiments, the plunger rod guide 152 may include a plurality of parts. For example, instead of the plunger rod guide 152 defining a single, monolithic component, the plunger rod guide 152 may include two or more parts that are coupled to each other, for example, by a clutch interface, to transfer energy from one part to another.

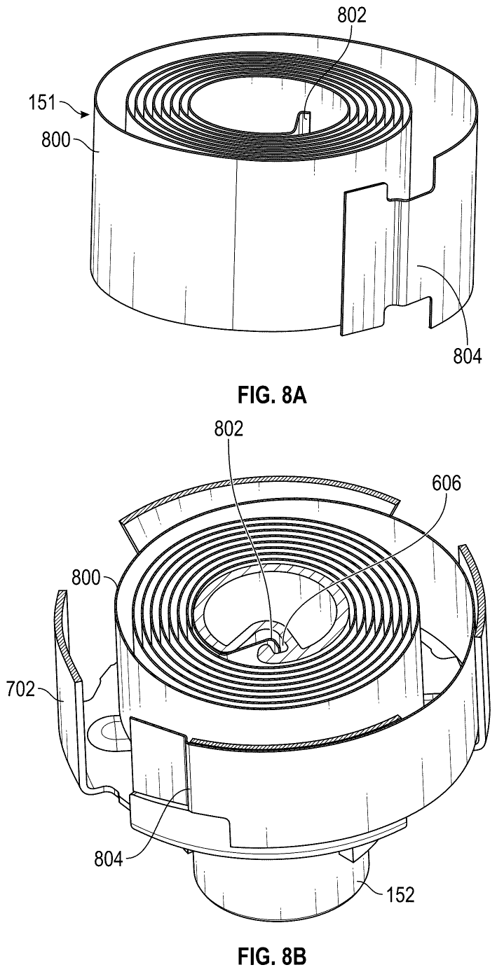

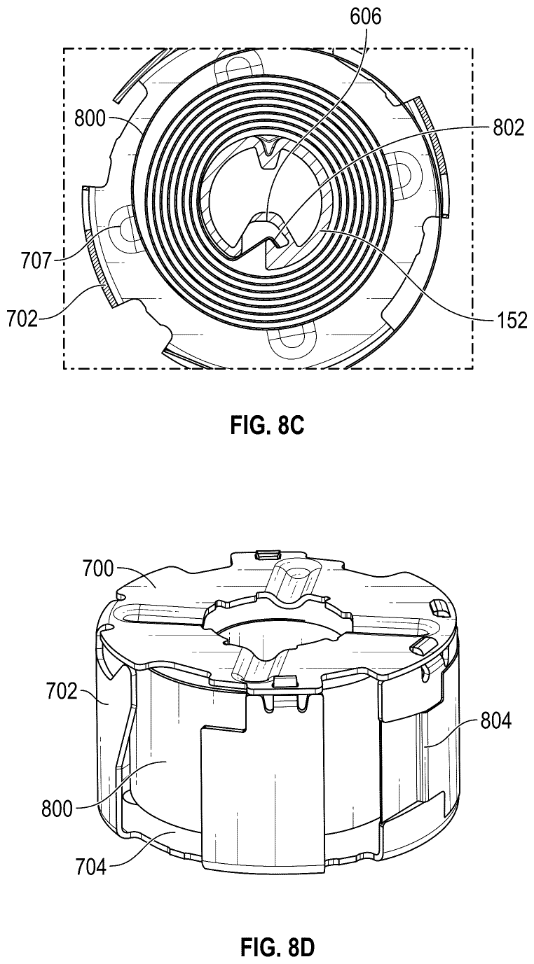

Referring now to FIGS. 8A-8D, the energy source 151 at least partially disposed within the casing 250 is described. The energy source 151 may be configured to store mechanical, electrical, and/or chemical energy. Upon activation, the energy source 151 may release or otherwise output this energy in order to generate the force needed for actuating one or more components of the drug delivery device 100. The energy source 151 may be a spring (e.g., a torsion spring, such as a helical torsion spring or a spiral torsion spring, a helical compression spring, a helical extension spring, etc.). In the embodiments of the present disclosure, the energy source 151 is defined by a spiral torsion spring, also sometimes referred to as a watch spring. In the pre-injection state, the energy source 151 may be retained in a wound or energized state. Accordingly, in the pre-assembled drive assembly 200, as illustrated in FIGS. 2A-2C and 3A-3C, the energy source 151 is retained in a wound or energized state. Activation of the drug delivery device 100 may release the energy source 151 so that it can unwind to a de- or less-energized state. The energy source 151 may output a biasing force in the pre-injection state and the injection state, and also optionally in the post-injection state. The biasing force output by the spiral torsion spring is a rotational force (i.e., a torque). The energy source 151 may exert a rotational force (i.e., a torque) on the plunger rod guide 152, which causes the plunger rod guide 152 and, consequently, the plunger 154 to rotate to expel the drug from the container 130.

The energy source 151 may be manufactured from a single band of metal 800. In some embodiments, the metal band 800 may be made of steel. The metal band 800 may include a first hook 802 at a first end thereof and a second hook 804 at a second end thereof. The first end of the metal band 800 may be bent to define the first hook 802, and the second end of the metal band 800 may be bent to define the second hook 804. As discussed above, the first hook 802 at the first end of the metal band 800 may be configured to engage with the recess 606 of the plunger rod guide 152 and lock the first end of the metal band 800 onto the plunger rod guide 152, as shown in FIGS. 8B and 8C. Once the first hook 802 is securely locked onto the recess 606 of the plunger rod guide 152, the metal band 800 may be wound around the plunger rod guide 152. Once the winding process is complete, the second hook 804 at the second end of the metal band 800 may engage with a sidewall 702 of the casing 250, as shown in FIG. 8D. For example, the second hook 804 may be removably attached to one of the sidewalls 702 of the casing 250. When attached, the portion of the metal band 800 adjacent to the second hook 804 may be flush with the outer diameter of the casing 250 such that the metal band 800 and/or the second hook 804 does not contact the inner surface of the rear housing portion 112b of the drug delivery device 100 when assembled into the drug delivery device 100.

As shown in FIGS. 8B-8D, the energy source 151 is at least partially disposed within the casing 250 and wound around the plunger rod guide 152. In some embodiments, during the winding process, the first end of the metal band 800 is inserted into the casing 250 from the side in a linear, unidirectional movement, and the first hook 802 is securely locked onto the recess 606 of the plunger rod guide 152. Afterwards, the metal band 800 is continuously fed into the casing 250 from the side thereof and wound around the plunger rod guide 152. Once the winding process is complete, the second hook 804 at the second end of the metal band 800 is attached to one of the sidewalls 702 of the casing 250 to secure the energy source 151 within the casing 250. As shown in FIGS. 2B and 3B, the plunger rod guide 152 and the energy source 151 are axially aligned with the longitudinal axis A.

In some embodiments, a sidewall 702 of the plurality of sidewalls 702 of the casing 250 may further include a tab 1100 (shown in FIG. 11). Additionally or alternatively, the second end of the metal band 800 may include a cavity 1102 (shown in FIG. 11). The cavity 1102 at the second end of the metal band 800 may engage the tab 1100 on the sidewall 702 of the casing 250 via, for example, a friction fit to securely lock the second end of the metal band 800 onto the casing 250.

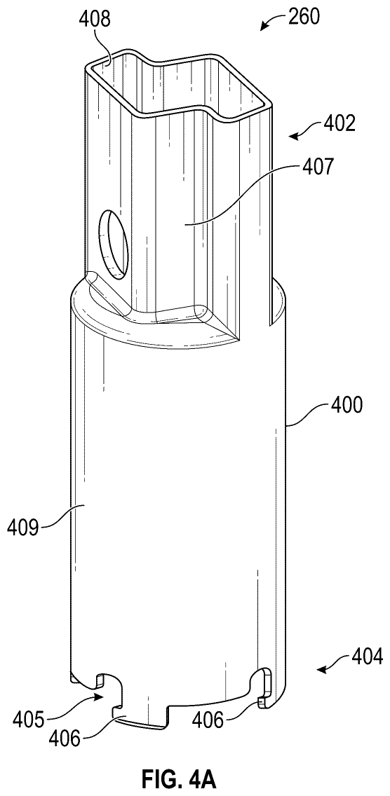

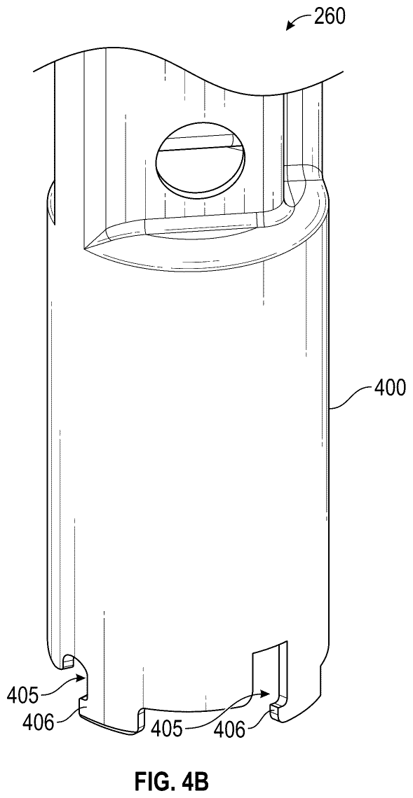

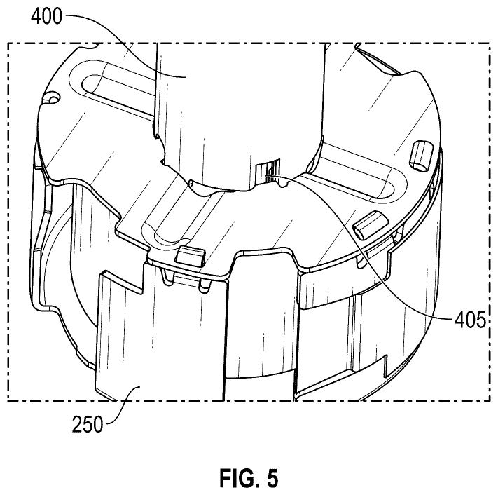

Referring now to FIGS. 4A, 4B, and 5, the lock component 260 is described. In some embodiments, the lock component 260 may be made of steel. The lock component 260 may have a hollow and generally cylindrical or tubular body portion 400 having a proximal end 402 and a distal end 404. The body portion 400 may include a distal body portion 409 extending proximally along the longitudinal axis from the distal end 404 thereof and a proximal body portion 407 extending proximally along the longitudinal axis from the distal body portion 409. When the lock component 260 is removably and/or selectively coupled with the casing 250, the lock component 260 may be axially aligned with the longitudinal axis A, as shown in FIG. 2B.

The lock component 260 may be configured and shaped to receive a portion of the plunger rod guide 152 therein. For example, as shown in FIGS. 2A-2C, the lock component 260 may be configured to receive a portion of the body 605 and the proximal portion 607 of the plunger rod guide 152 therein, at least when the lock component 260 is coupled with the casing 250. In some embodiments, an inner diameter of the body portion 400 of the lock component 260 may be larger than an outer diameter of the body 605 of the plunger rod guide 152 such that the plunger rod guide 152 and the lock component 260 do not contact each other; whereas, in other embodiments, the two components may be in contact or allowed to contact each other.

The distal body portion 409 and the proximal body portion 407 may be hollow and may define a cavity 408 therein. Similar to the proximal portion 607 of the plunger rod guide 152, the proximal body portion 407 of the lock component 260 may have a non-circular cross-section that is perpendicular to the longitudinal axis. While the distal body portion 409 of the lock component 400 may have a generally circular cross-section, the proximal body portion 407 may have a cross-section that is shaped like a “Z” (i.e., Z-shaped). The proximal body portion 407 having a Z-shaped cross-section may be shaped to accommodate the proximal portion 607 of the plunger rod guide 152 therein. Accordingly, the shape of the proximal body portion 407 of the lock component 260 may complement the shape of the proximal portion 607 of the plunger rod guide 152.

In some embodiments, the lock component 260 may further include at least one hook 406 adjacent to the distal end 404 thereof. In some embodiments, the lock component 260 may include a plurality of hooks 406 adjacent to the distal end 404 thereof. In yet another embodiment, the plurality of hooks 406 may be positioned along a circumference of the distal end 404 of the distal body portion 409. As shown in FIG. 5, the plurality of hooks 406 may each be configured to removably and/or selectively engage a respective one of the cut-out portions 706 in the lid 700 of the casing 250 to removably and/or selectively secure the lock component 260 onto the proximal end 701 of the casing 250.

When the lock component 260 is coupled with the casing 250, at least a portion of the plunger rod guide 152 may already be disposed within the casing 250, and the energy source 151 may already be wound around the plunger rod guide 152 in an energized state, as shown in FIGS. 3A-3C. In order to maintain the energy source 151 in the wound and energized state and to prevent the energy source 151 from unwinding, the lock component 260 may be removably and/or selectively coupled to the proximal end 701 of the casing 250. To removably and/or selectively couple the lock component 260 onto the casing 250, the lock component 260 may be positioned over the proximal portion 607 of the plunger rod guide 152 such that at least a portion of the body 605, as well as the proximal portion 607, of the plunger rod guide 152 is accommodated within the lock component 260, as shown in FIGS. 2A-2C. To secure the lock component 260 onto the casing 250, the hooks 406 may engage a respective one of the cut-out portions 705 in the lid 700 of the casing 250. When the lock component 260 is removably and/or selectively locked onto the casing 250 and the plunger rod guide 152 is accommodated within the lock component 260, the Z-shaped proximal body portion 407 of the lock component 260 prevents the Z-shaped proximal portion 607 of the plunger rod guide 152 from rotating and, thus, rotationally and radially restrains movement of the plunger rod guide 152. Accordingly, when the lock component 260 is removably and/or selectively locked onto the casing 250, the lock component 260 prevents rotational movement of the plunger rod guide 152 relative to the casing 250, thereby preventing the energy source 151 from unwinding and locking the energy source 151 in its energized state. The lock component 260 rotationally locks the plunger rod guide 152 relative to the casing 250 and maintains the energy source 151 in the energized state until the lock component 260 is disengaged and removed from the casing 250 after the drive assembly 200 is axially assembled into the drug delivery device 100. In some embodiments, the hooks 406 may securely engage a respective one of the cut-out portions 705 in the lid 700 such that, even when the lock component 260 and/or the casing 250 are being moved at a high speed or acceleration, the hooks 406 are able to grip onto a respective one of the cut-out portions 705.

Additionally, the lock component 260 may include one or more windows 405 adjacent to the distal end 404 of the distal body portion 409. The one or more windows 405 may allow viewing and/or visual inspection to ensure the hooks 406 are securely locked into the cut-out portions 706 in the lid 700 of the casing 250 when coupling the lock component 260 onto the casing 250, and/or to ensure the hooks 406 are disengaged from the cut-out portions 706 when de-coupling the lock component 260 from the casing 250. In addition, the one or more windows 405 may allow viewing and/or visual inspection to ensure the axial position of the energy source 151 relative to the plunger rod guide 152 and/or the casing 250 is within a predetermined range.

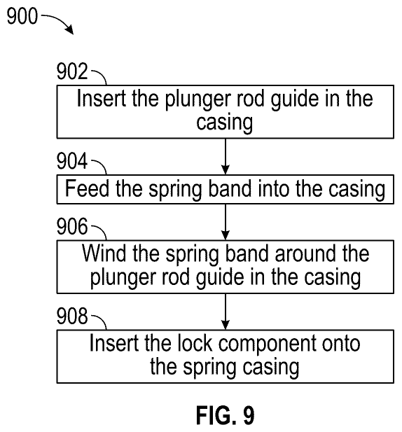

Referring now to FIGS. 9 and 12-23, an exemplary method 900 of assembling the drive assembly 200 is described in detail. Prior to step 902, the method 900 may comprise providing the casing 250 shown in FIGS. 7A-7D. In some embodiments, the method 900 of assembling the drive assembly 200 may be completely automated by a machine. Accordingly, each step of the method 900 may be automated, thereby increasing the efficiency of the assembly process and ensuring accuracy and quality of the drive assembly 200.

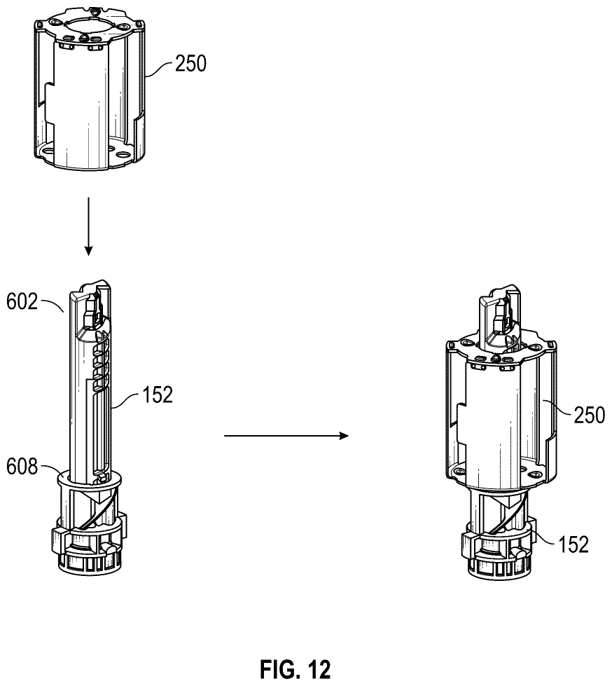

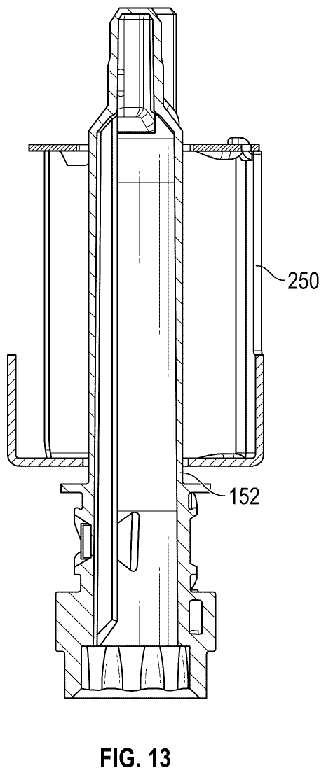

The method 900 begins at step 902 at which the plunger rod guide 152 (FIG. 6) is inserted in the casing 250. As shown in FIG. 12 and as discussed above, inserting the plunger rod guide 152 in the casing 250 may comprise axially inserting the proximal end 602 of the plunger rod guide 152 through the cavity 710 (FIG. 7D) on the bottom surface 704 of the casing 250 and through the cavity 710 (FIG. 7C) on the lid 700 of the casing 250 such that at least a portion of the plunger rod guide 152 is disposed within the casing 250 (also shown in FIGS. 2A-2C and 3A-3C). In other embodiments, inserting the plunger rod guide 152 in the casing 250 may comprise axially positioning the casing 250 onto the plunger rod guide 152. When the plunger rod guide 152 is inserted in the casing 250, the flange 608 (FIG. 6) may be positioned distal to the bottom surface 704 (FIGS. 7B and 7D) of the casing 250, as shown in FIGS. 2A-2C and 3A-3C). FIG. 13 further illustrates a cross-sectional view of the plunger rod guide 152 after the plunger rod guide 152 is inserted in the casing 250. Additionally, when the plunger rod guide 152 is inserted in the casing 250, the plunger rod guide 152 may be rotationally positioned relative to the casing 250 such that the recess 606 (FIG. 6) of the plunger rod guide 152 is aligned with an opening in the plurality of sidewalls 702 of the casing 250.

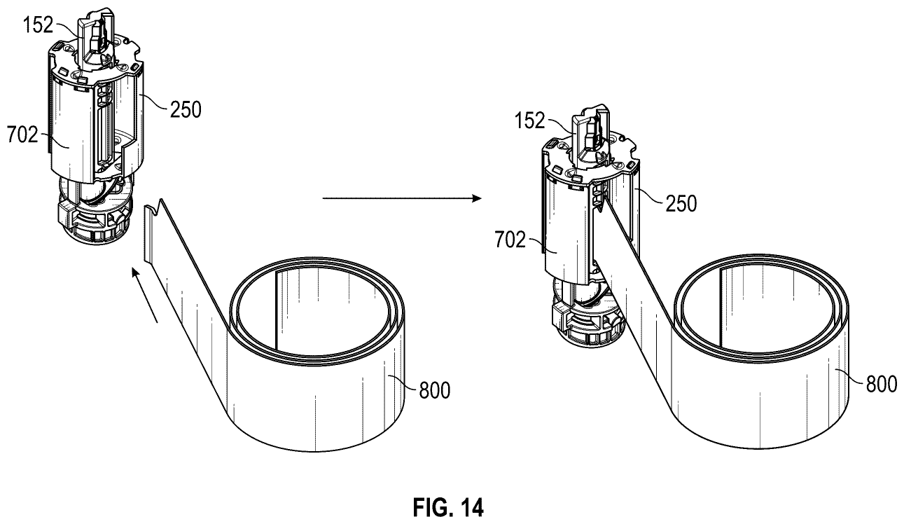

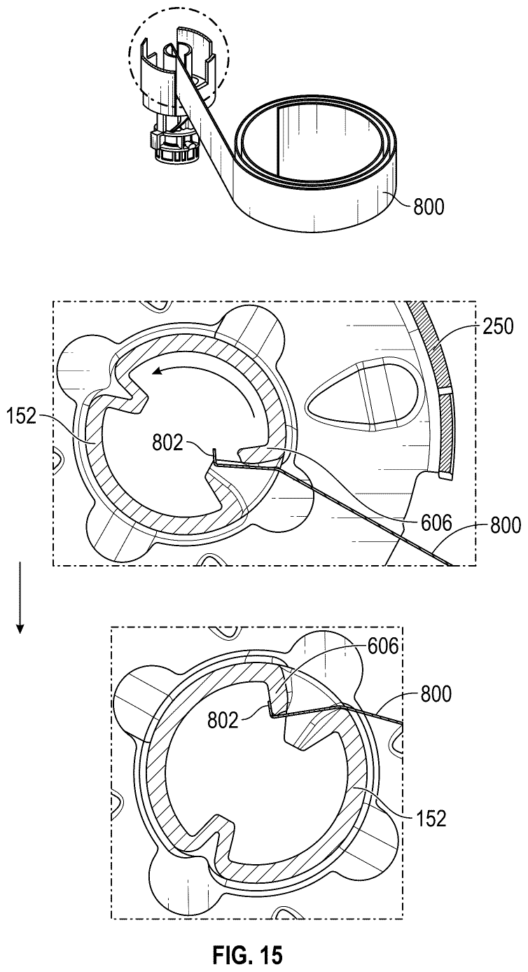

Once the plunger rod guide 152 is inserted in the casing 250, the method 900 may proceed to step 904, at which the metal band 800 of the energy source 151 is fed into the casing 250 through an opening in the plurality of sidewalls 702 of the casing 250 and onto the plunger rod guide 152 within the casing 250, as illustrated in FIG. 14. Feeding the metal band 800 into the casing 250 may comprise feeding the first end of the metal band 800 into the casing 250 from the side of the casing 250 in a linear, unidirectional movement, and locking the first hook 802 at the first end of the metal band 800 onto the recess 606 of the plunger rod guide 152. For example, as shown in FIG. 15, when the first end of the metal band 800 is fed into the casing 250, the first hook 802 at the first end of the metal band 800 may engage and lock onto the recess 606 of the plunger rod guide 152 such that the first end of the metal band 800 can no longer disengage from the plunger rod guide 152.

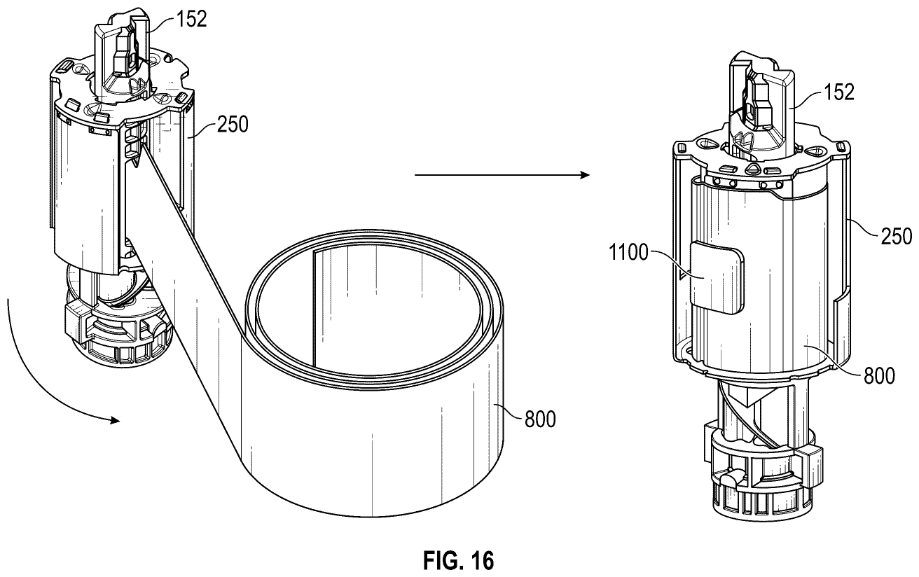

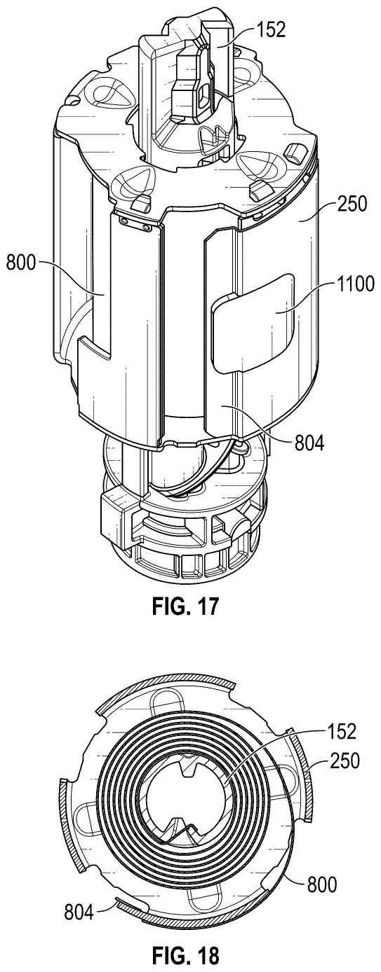

Once the first end of the metal band 800 is fed into the casing 250, the method 900 continues to step 906, at which the metal band 800 is continuously fed into the casing 250 from the side thereof and wound around the plunger rod guide 152 in the casing 250 to form the energy source 151. For example, as illustrated in FIG. 16, after the first end of the metal band 800 is locked onto the recess 606 of the plunger rod guide 152 within the casing 250, a torque may be applied to the plunger rod guide 152 to rotate the plunger rod guide 152 in the counter-clockwise direction until the metal band 800 is completely wound onto the plunger rod guide 152. During the winding process, the axial and radial position of the plunger rod guide 152, the casing 250, and the metal band 800 may be constrained by the machine to prevent axial displacement of the metal band 800 and ensure accurate positioning of the metal band 800 relative to the plunger rod guide 152. Once the winding process is complete, the second hook 804 (FIG. 11) at the second end of the metal band 800 is attached to one of the sidewalls 702 of the casing 250 to secure the energy source 151 within the casing 250 in its wound, energized state. For example, as further illustrated in FIGS. 17 and 18, the second hook 804 of the metal band 800 may engage the tab 1100 on the casing 250 to secure the metal band 800 within the casing 250 in its wound, energized state. In some embodiments, the second hook 804 of the metal band 800 may be inserted under the tab 1100 on the casing 250. The engagement between the second hook 804 and the tab 1100 (in addition to the engagement between the first hook 802 and the recess 606 of the plunger rod guide 152) may ensure the energy source 151 is maintained in its wound, energized state within the casing 250.

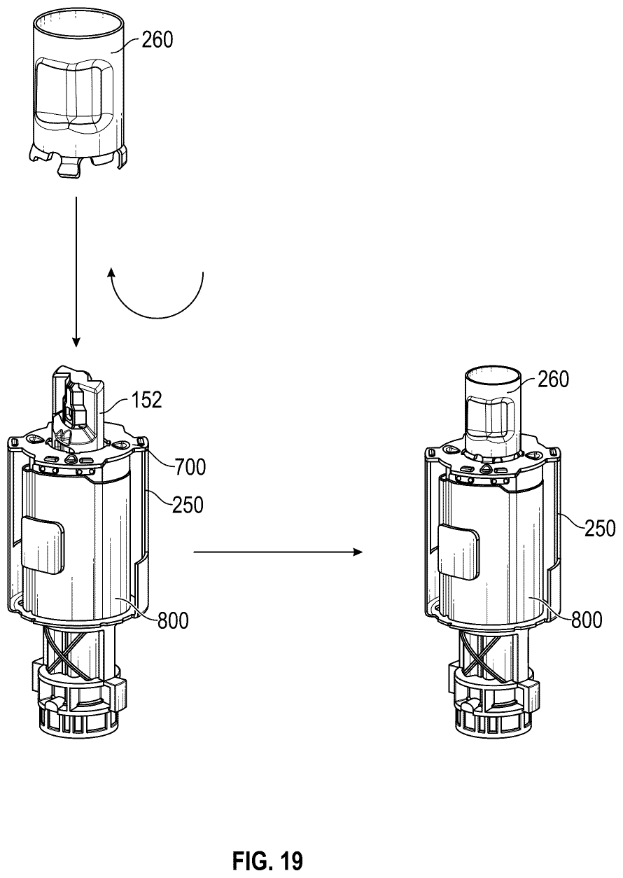

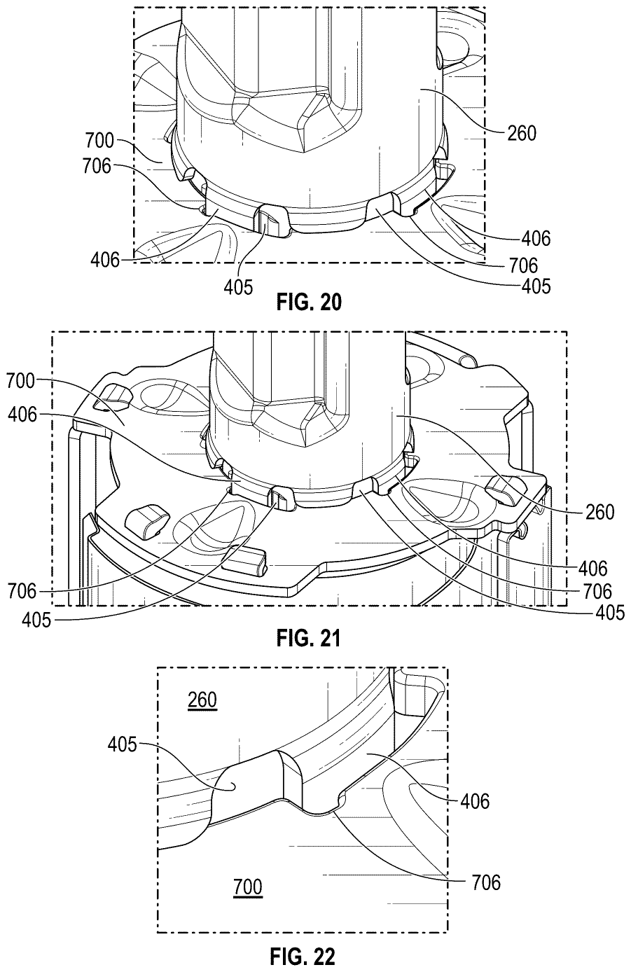

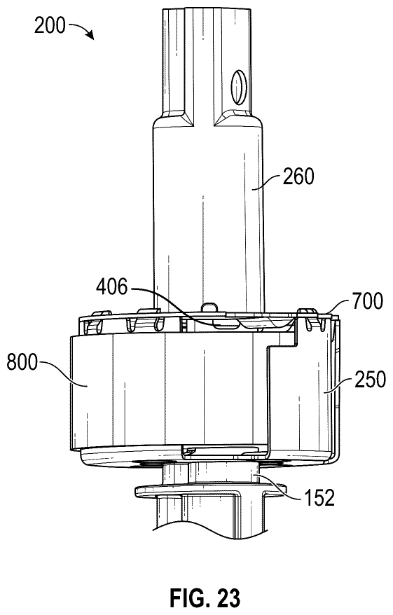

The method further proceeds to step 908, at which the lock component 260 (FIGS. 4A-4B and 5) is inserted onto the casing 250. Inserting the lock component 260 onto the casing 250 may comprise removably and/or selectively coupling the lock component 260 onto the lid 700 of the casing 250. For example, as shown in FIG. 19, the lock component 260 may be axially inserted onto the casing 250 and rotated, for example in a clockwise direction, until the lock component 260 is locked onto the lid 700 of the casing 250. As shown in FIGS. 20-22, when the lock component 260 is inserted into the casing 250 and rotated in the clockwise direction, the hooks 406 (also shown in FIG. 4A) adjacent to the distal end 404 of the lock component 260 may engage with respective one of the cut-out portions 706 in the lid 700 of the casing 250 to removably and/or selectively secure the lock component 260 onto the lid 700 of the casing 250. As shown in more detail in FIGS. 22 and 23, when the hooks 406 of the lock component 260 engage the respective cut-out portions 706 in the lid 700 of the casing 250 to lock the lock component 260 onto the casing 250, at least a portion of each hook 406 may be positioned under the lid 700 of the casing 250 such that the lock component 260 cannot be axially pulled off the casing 250. Accordingly, to remove the lock component 260 from the casing 250, the lock component 260 will first need to be rotated, for example in a counter-clockwise direction, to disengage the hooks 406 from the respective cut-out portions 706 in the lid 700 and axially lifted from the casing 250.

When the lock component 260 is coupled with the casing 250, the lock component 260 may be positioned over the proximal portion 607 of the plunger rod guide 152 such that at least a portion of the body 605, as well as the proximal portion 607, of the plunger rod guide 152 is accommodated within the lock component 260, as shown in FIGS. 2A-2C. When the lock component 260 is removably and/or selectively locked onto the casing 250 and the plunger rod guide 152 is accommodated within the lock component 260, the Z-shaped proximal body portion 407 of the lock component 260 prevents the Z-shaped proximal portion 607 of the plunger rod guide 152 from rotating and, thus, rotationally and radially restrains movement of the plunger rod guide 152. Accordingly, when the lock component 260 is removably and/or selectively locked onto the casing 250, the lock component 260 prevents rotational movement of the plunger rod guide 152 relative to the casing 250, thereby preventing the energy source 151 from unwinding and locking the energy source 151 in its wound, energized state. The lock component 260 rotationally locks the plunger rod guide 152 relative to the casing 250 and maintains the energy source 151 in the wound, energized state until the lock component 260 is disengaged and removed from the casing 250 after the drive assembly 200 is axially assembled into the drug delivery device 100.

In some embodiments, the method 900 may further comprise axially assembling the drive assembly 200 into a drug delivery device, such as the drug delivery device 100. When axially assembling the drive assembly 200 into the drug delivery device 100, the lock component 260 is coupled with the casing 250 so as to rotationally lock the plunger rod guide 152 relative to the casing 250 and maintain the energy source 151 in its wound, energized state. Because the drive assembly 200 already includes the energy source 151 disposed within the casing 250 and the plunger rod guide 152 operably coupled to the casing 250, axially assembling the drive assembly 200 into the drug delivery device 100 can be performed in a single step. In some embodiments, the drive assembly 200 is axially assembled into the rear housing portion 112b of the drug delivery device 100. Once the drive assembly 200 is assembled into the drug delivery device 100, the lock component 260 can be removed from the casing 250 by disengaging the hooks 406 from the respective cut-out portion 706. The steps of axially assembling the drive assembly 200 into the drug delivery device 100 and removing the lock component 260 from the casing 250 can be fully automated to ensure precise placement of the drive assembly 200 in the drug delivery device 100 and to increase efficiency of the assembly process.

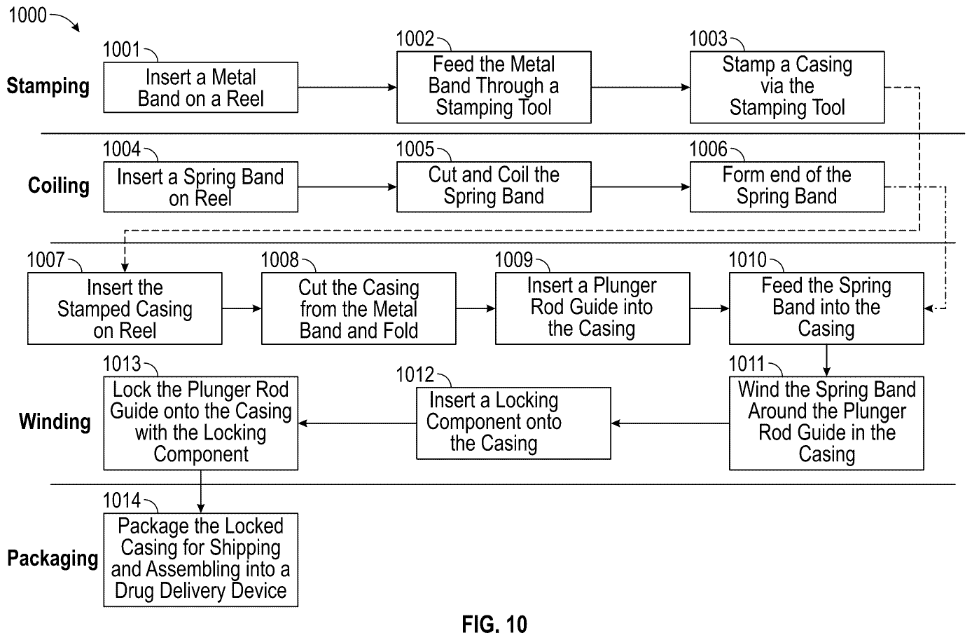

FIG. 10 illustrates an exemplary method 1000 of manufacturing the drive assembly 200 and assembling the drive assembly 200 into the drug delivery device 100, in accordance with various embodiments of the present disclosure. In some embodiments, the method 1000 of manufacturing and assembling the drive assembly 200 may be mostly or completely automated. Accordingly, each step of the method 1000 may be automated, thereby increasing the efficiency of the manufacturing and assembly processes and ensuring accuracy and quality of the drive assembly 200.

The method 1000 begins at step 1001, at which a sheet of metal is inserted on a reel. At step 1002, the sheet of metal is fed through a stamping tool and, subsequently, at step 1003, the stamping tool is used to stamp the metal sheet to form the casing 250. In some embodiments, the casing 250 may be stamped, via the stamping tool, with one or more features for automated gripping. Additionally, or alternatively, the casing 250 may be stamped with the tab 1100 (FIG. 11). The stamped metal sheet is, then, inserted on a reel at step 1007. In some embodiments, at step 1008, the stamped metal sheet may be cut using a cutting tool to form the bottom surface 704, the sidewalls 702, and the lid 700 of the casing 250 that are integrally formed from the single sheet of metal. When the stamped metal sheet is cut to form the bottom surface 704, the sidewalls 702, and the lid 700, the casing 250 may be in its flat configuration, where the bottom surface 704, the sidewalls 702, and the lid 700 are substantially flat. Afterwards, the metal sheet may be folded to form the casing 250 having a cylindrical shape. When the metal sheet is folded to form the casing 250 having a cylindrical shape, the casing 250 may be in its assembled configuration, where the bottom surface 704, the sidewalls 702, and the lid 700 are folded to form the cylindrical body portion of the casing 250. For example, the sidewalls 702 may be folded up to define the cylindrical body portion of the casing 250, and the lid 700 may be folded over the sidewalls 702 to form an overlid. FIGS. 7A-7D illustrate the casing 250 in its assembled configuration after steps 1001, 1002, 1003, 1007, and 1008 are completed. In some embodiments, when the casing 250 is in its assembled configuration, the protrusions 705 extending from the sidewalls 702 of the casing 250 may be inserted through the respective openings 708 on the lid 700 and clinched to lock the lid 700 of the casing 250 in a closed configuration.

Separately, at step 1004, a metal band, such as the metal band 800, is inserted on a reel to manufacture the energy source 151. At step 1005, the metal band 800 is cut and coiled. Additionally, at step 1006, the ends of the metal band 800 is formed. In some embodiments, forming the ends of the metal band 800 may comprise bending the first end of the metal band 800 to form the first hook 802 and bending the second end of the metal band 800 to form the second hook 804. In some embodiments, forming the ends of the metal band 800 may also comprise cutting the cavity 1102 (FIG. 11) proximate the second end of the metal band 800.

The method 1000 now proceeds to step 1009, at which the plunger rod guide 152 (FIG. 6) is inserted in the casing 250 that is manufactured at steps 1001, 1002, 1003, 1007, and 1008. As discussed above, inserting the plunger rod guide 152 in the casing 250 may comprise axially inserting the proximal end 602 of the plunger rod guide 152 through the cavity 710 (FIG. 7D) on the bottom surface 704 of the casing 250 and through the cavity 710 (FIG. 7C) on the lid 700 of the casing 250 such that at least a portion of the plunger rod guide 152 is disposed within the casing 250 (also shown in FIGS. 2A-2C and 3A-3C). In other embodiments, inserting the plunger rod guide 152 in the casing 250 may comprise axially positioning the casing 250 onto the plunger rod guide 152. When the plunger rod guide 152 is inserted in the casing 250, the flange 608 (FIG. 6) may be positioned distal to the bottom surface 704 (FIGS. 7B and 7D) of the casing 250, as shown in FIGS. 2A-2C and 3A-3C). Additionally, when the plunger rod guide 152 is inserted in the casing 250, the plunger rod guide 152 may be rotationally positioned relative to the casing 250 such that the recess 606 (FIG. 6) of the plunger rod guide 152 is aligned with an opening in the plurality of sidewalls 702 of the casing 250.

Once the plunger rod guide 152 is inserted in the casing 250, the method 1000 may proceed to step 1010, at which the metal band 800 manufactured at steps 1004, 1005, and 1006 is fed into the casing 250 through an opening in the plurality of sidewalls 702 of the casing 250 and onto the plunger rod guide 152 within the casing 250. Feeding the metal band 800 into the casing 250 may comprise feeding the first end of the metal band 800 into the casing 250 from the side of the casing 250 in a linear, unidirectional movement, and locking the first hook 802 at the first end of the metal band 800 onto the recess 606 of the plunger rod guide 152. For example, as shown in FIG. 15, when the first end of the metal band 800 is fed into the casing 250, the first hook 802 at the first end of the metal band 800 may engage and lock onto the recess 606 of the plunger rod guide 152 such that the first end of the metal band 800 can no longer disengage from the plunger rod guide 152.

Once the first end of the metal band 800 is fed into the casing 250, the method 1000 continues to step 1011, at which the metal band 800 is continuously fed into the casing 250 from the side thereof and wound around the plunger rod guide 152 in the casing 250 to form the energy source 151. For example, as illustrated in FIG. 16, after the first end of the metal band 800 is locked onto the recess 606 of the plunger rod guide 152 within the casing 250, a torque may be applied to the plunger rod guide 152 to rotate the plunger rod guide 152 in the counter-clockwise direction until the metal band 800 is completely wound onto the plunger rod guide 152. During the winding process, the axial and radial position of the plunger rod guide 152, the casing 250, and the metal band 800 may be constrained by the machine to prevent axial displacement of the metal band 800 and ensure accurate positioning of the metal band 800 relative to the plunger rod guide 152. Once the winding process is complete, the second hook 804 (FIG. 11) at the second end of the metal band 800 is attached to one of the sidewalls 702 of the casing 250 to secure the energy source 151 within the casing 250 in its wound, energized state. For example, as further illustrated in FIGS. 17 and 18, the second hook 804 of the metal band 800 may engage the tab 1100 on the casing 250 to secure the metal band 800 within the casing 250 in its wound, energized state. In some embodiments, the second hook 804 of the metal band 800 may be inserted under the tab 1100 on the casing 250. The engagement between the second hook 804 and the tab 1100 (in addition to the engagement between the first hook 802 and the recess 606 of the plunger rod guide 152) may ensure the energy source 151 is maintained in its wound, energized state within the casing 250.

The method further proceeds to step 1012, at which the lock component 260 (FIGS. 4A-4B and 5) is inserted onto the casing 250. Inserting the lock component 260 onto the casing 250 may comprise removably and/or selectively coupling the lock component 260 onto the lid 700 of the casing 250. For example, as shown in FIG. 19, the lock component 260 may be axially inserted onto the casing 250 and rotated, for example in a clockwise direction, until the lock component 260 is locked onto the lid 700 of the casing 250. As shown in FIGS. 20-22, when the lock component 260 is inserted into the casing 250 and rotated in the clockwise direction, the hooks 406 (also shown in FIG. 4A) adjacent to the distal end 404 of the lock component 260 may engage with respective one of the cut-out portions 706 in the lid 700 of the casing 250 to removably and/or selectively secure the lock component 260 onto the lid 700 of the casing 250. As shown in more detail in FIGS. 22 and 23, when the hooks 406 of the lock component 260 engage the respective cut-out portions 706 in the lid 700 of the casing 250 to lock the lock component 260 onto the casing 250, at least a portion of each hook 406 may be positioned under the lid 700 of the casing 250 such that the lock component 260 cannot be axially pulled off the casing 250. Accordingly, to remove the lock component 260 from the casing 250, the lock component 260 will first need to be rotated, for example in a counter-clockwise direction, to disengage the hooks 406 from the respective cut-out portions 706 in the lid 700 and axially lifted from the casing 250.

When the lock component 260 is coupled with the casing 250, at step 1013, the lock component 260 may be configured to lock the plunger rod guide 152 onto the casing 250. In particular, the lock component 260 may be positioned over the proximal portion 607 of the plunger rod guide 152 such that at least a portion of the body 605, as well as the proximal portion 607, of the plunger rod guide 152 is accommodated within the lock component 260, as shown in FIGS. 2A-2C. When the lock component 260 is removably and/or selectively locked onto the casing 250 and the plunger rod guide 152 is accommodated within the lock component 260, the Z-shaped proximal body portion 407 of the lock component 260 prevents the Z-shaped proximal portion 607 of the plunger rod guide 152 from rotating and, thus, rotationally and radially restrains movement of the plunger rod guide 152. Accordingly, when the lock component 260 is removably and/or selectively locked onto the casing 250, the lock component 260 prevents rotational movement of the plunger rod guide 152 relative to the casing 250, thereby preventing the energy source 151 from unwinding and locking the energy source 151 in its wound, energized state. The lock component 260 rotationally locks the plunger rod guide 152 relative to the casing 250 and maintains the energy source 151 in the wound, energized state until the lock component 260 is disengaged and removed from the casing 250 after the drive assembly 200 is axially assembled into the drug delivery device 100.

At step 1014, the locked casing 250 defining the drive assembly 200 shown in FIGS. 2A-2C is packaged for shipping and assembling into the drug delivery device 100. For example, at step 1014, the locked casing 250 may be packaged in an upright position to increase the number of parts that can be shipped on each pallet per shipper. During the subsequent assembly process, the drive assembly 200 may be axially assembled into the drug delivery device 100 while the lock component 260 is coupled with the casing 250 so as to rotationally lock the plunger rod guide 152 relative to the casing 250 and maintain the energy source 151 in its wound, energized state. Because the drive assembly 200 already includes the energy source 151 disposed within the casing 250 and the plunger rod guide 152 operably coupled to the casing 250, axially assembling the drive assembly 200 into the drug delivery device 100 can be performed in a single step. In some embodiments, the drive assembly 200 is axially assembled into the rear housing portion 112b of the drug delivery device 100. Once the drive assembly 200 is assembled into the drug delivery device 100, the lock component 260 can be removed from the casing 250 by disengaging the hooks 406 from the respective cut-out portion 706. The steps of axially assembling the drive assembly 200 into the drug delivery device 100 and removing the lock component 260 from the casing 250 can be fully automated to ensure precise placement of the drive assembly 200 in the drug delivery device 100 and to increase efficiency of the assembly process.

As will be recognized, the devices and methods according to the present disclosure may have one or more advantages relative to conventional technology, any one or more of which may be present in a particular embodiment in accordance with the features of the present disclosure included in that embodiment. Other advantages not specifically listed herein may also be recognized as well.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity® (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. In some embodiments, the drug delivery device may contain or be used with Bemarituzumab, a monoclonal antibody that inhibits fibroblast growth factor receptor 2b (FGFR2b) for the treatment of advanced Gastric and Gastroesophageal Junction (GEJ) cancers. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF α monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv^{T}® (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 206 (nivolumab), a biosimilar candidate to OPDIVO®, or another product containing a monoclonal antibody that targets the PD-1 protein on T cells. In some embodiments, the drug delivery device may contain or be used with ABP 234 (pembrolizumab), a biosimilar candidate to KEYTRUDA®, or another product containing a monoclonal antibody that binds to the PD-1 protein on immune cells. In some embodiments, the drug delivery device may contain or be used with ABP 692 (ocrelizumab), a biosimilar candidate to OCREVUS®, or another product containing a humanized anti-CD20 monoclonal antibody. In some embodiments, the drug delivery device may contain or be used with daxdilimab, a human monoclonal antibody against ILT7 or another product. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with Pavblu®, a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Imdelltra® (tarlatamab-dlle), an anti-delta-like ligand 3 (DLL3 ) x anti-CD3 bispecific T cell engager (BiTE) molecule, or another product containing tarlatamab-dlle for the treatment of small cell lung cancer. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Lumakras® (sotorasib), a KRAS^{G12C} small molecule inhibitor, or another product containing a KRAS^{G12C} small molecule inhibitor. In some embodiments, the drug delivery device may contain or be used with Tezaspire® (tezepelumab-ekko), a human monoclonal antibody that inhibits the action of thymic stromal lymphopoietin (TSLP), or another product containing a human monoclonal antibody that inhibits the action of TSLP. In some embodiments, the drug delivery device may contain or be used with Tavneos® (avacopan), a complement 5a receptor 1 (C5aR1) antagonist that inhibits the effects of C5a. In some embodiments, the drug delivery device can contain or be used with Tepezza® (teprotumumab-trbw), a human monoclonal antibody against insulin-like growth factor-1 receptor (IGF-1R), or another product containing a human monoclonal antibody against IGF-1R. In some embodiments, the drug delivery device can contain or be used with Uplizna® (inebilizumab-cdon), a humanized monoclonal antibody that binds to the B cell-specific surface antigen CD19, or another product containing a humanized monoclonal antibody that binds to the B cell-specific surface antigen CD19. In some embodiments, the drug delivery device may contain or be used with rocatinlimab (AMG 451), a human anti-OX40 monoclonal antibody that is expressed on activated T cells and blocks OX40 to inhibit and/or reduce the number of OX40 pathogenic T cells that are responsible for driving system and local atopic dermatitis inflammatory responses. In some embodiments, the drug delivery device may contain or be used with ordesekimab (AMG 714), a human monoclonal antibody that binds to Interleukin-15 (IL-15) or another product containing a human monoclonal antibody that binds to Interleukin-15 (IL-15). In some embodiments, the drug delivery device may contain or be used with olpasiran (AMG 890), a small interfering RNA (siRNA) that lowers lipoprotein(a), also known as Lp(a), or another product containing a small interfering RNA (siRNA) that lowers lipoprotein(a). In some embodiments, the drug delivery device may contain or be used with Wezlana®/Wezenla™ (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 104, or another product containing a human anti-TSLP Fab. In some embodiments, the drug delivery device may contain or be used with AMG 193, or another product containing a small molecule methylthioadenosine (MTA) cooperative protein arginine methyltransferase 5 (PRMT5) inhibitor. In some embodiments, the drug delivery device may contain or be used with AMG 329, or another product containing a human monoclonal antibody that binds and neutralizes the function of the FLT3-ligand. In some embodiments, the drug delivery device may contain or be used with AMG 732, or another product containing a monoclonal antibody against insulin-like growth factor-1 receptor (IGF-1R). In some embodiments, the drug delivery device may contain or be used with AMG 305, or another product containing dual-targeting bispecific T cell engager (BiTE) molecule against P-cadherin (CDH3), mesothelin (MSLN) and CD3. In some embodiments, the drug delivery device may contain or be used with AMG 355, or another product containing an anti-CCR8 monoclonal antibody. In some embodiments, the drug delivery device may contain or be used with AMG 378, or another product containing a small molecule for the treatment of ulcerative colitis. In some embodiments, the drug delivery device may contain or be used with AMG 410, or another product containing a small molecule for the treatment of solid tumors. In some embodiments, the drug delivery device may contain or be used with AMG 513, or another product containing a molecule for the treatment of obesity. In some embodiments, the drug delivery device may contain or be used with AMG 691, or another product containing a monoclonal antibody for the treatment of asthma. In some embodiments, the drug delivery device may contain or be used with AMG 160, or another product that contains a half-life extended (HLE) anti-prostate-specific membrane antigen (PSMA) x anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with MariTide™ (AMG 133), or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1 x IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33 x anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3) x anti-CD 3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP x 4-1BB-targeting DARPin® biologic under investigation as a treatment for solid tumors. In some embodiments, the drug delivery device may contain or be used with AMG 509 (xaluritamig) or another product containing a bivalent T-cell engager and is designed using XmAb® 2+1 technology. In some embodiments, the drug delivery device may contain or be used with AMG 562 or another product containing a half-life extended (HLE) CD19 x CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3 x epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33 x anti-CD3 BiTE® (bispecific T cell engager) construct. In some embodiments, the drug delivery device may contain or be used with AMG 701 or another product containing a half-life extended (HLE) anti-B-cell maturation antigen (BCMA) x anti-CD3 BiTE® (bispecific T cell engager) construct. In some embodiments, the drug delivery device may contain or be used with AMG 757 or another product containing a half-life extended (HLE) anti-delta-like ligand 3 (DLL3) x anti-CD 3 BiTE® (bispecific T cell engager) construct. In some embodiments, the drug delivery device may contain or be used with AMG 910 or another product containing a half-life extended (HLE) epithelial cell tight junction protein claudin 18.2 x CD3 BiTE® (bispecific T cell engager) construct. In some embodiments, the drug delivery device may contain or be used with Dazodalibep, a fusion protein binding CD40L on T cells to block their interaction with CD40-expressing B cells.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above-described embodiments without departing from the spirit and scope of the invention(s) disclosed herein, and that such modifications, alterations, and combinations are to be viewed as being within the ambit of the inventive concept(s).

1. A drive assembly for a drug delivery device, comprising:
a casing having a proximal end, a distal end, and a longitudinal axis extending between the proximal end and the distal end;
a plunger rod guide operably coupled to the casing;
a spring at least partially disposed within the casing and wound around at least a portion of the plunger rod guide in an energized state, the spring being configured to exert a force on the plunger rod guide that causes the plunger rod guide to rotate; and
a lock component selectively coupled with the casing and configured to receive at least a portion of the plunger rod guide therein, wherein the lock component is configured to prevent rotational movement of the plunger rod guide relative to the casing when the lock component is coupled with the casing to lock the spring in the energized state.

2. The drive assembly of claim 1, wherein the casing includes a bottom surface, a plurality of sidewalls extending from the bottom surface and defining a cylindrical body portion, and a lid, and wherein the bottom surface, the plurality of sidewalls, and the lid are integrally formed from a single sheet of metal.

3. (canceled)

4. (canceled)

5. The drive assembly of claim 2, wherein:
the casing further includes at least one opening formed through the lid,
at least one sidewall of the plurality of sidewalls includes a protrusion extending from the at least one sidewall, and
the protrusion is configured to clinch through the at least one opening on the lid to lock the lid in a closed configuration.

6. The drive assembly of claim 2, wherein the casing further includes a cavity formed through the bottom surface and the lid, and wherein at least a portion of the plunger rod guide is disposed through the cavity.

7. The drive assembly of claim 6, wherein the lid includes one or more cut-out portions, and wherein the lock component is configured to selectively engage the one or more cut-out portions to lock the spring in the energized state.

8. The drive assembly of claim 7, wherein the lock component has a proximal end, a distal end, and at least one hook adjacent to the distal end, wherein the at least one hook is configured to selectively engage the one or more cut-out portions of the lid to prevent rotational movement of the plunger rod guide relative to the casing.

9. (canceled)

10. (canceled)

11. The drive assembly of claim 1, wherein the spring includes a torsion spring and is formed from a single metal band, the metal band having a first end and a second end.

12. The drive assembly of claim 11, wherein the first end of the metal band includes a first hook and the second end of the metal band includes a second hook, wherein the first hook is configured to engage at least a portion of the plunger rod guide to lock the first end of the metal band onto the plunger rod guide, and wherein the second hook is configured to engage a sidewall of the casing.

13.-33. (canceled)

34. A method of assembling a drive assembly for a drug delivery device, the method comprising:
providing a casing having a bottom surface, a plurality of sidewalls extending from the bottom surface and defining a cylindrical body portion, and a lid;
inserting at least a portion of a plunger rod guide through a cavity formed through the bottom surface and the lid of the casing;
feeding a metal band into the casing through an opening in the plurality of sidewalls;
winding the metal band around a portion of the plunger rod guide within the casing to form a spring; and
selectively coupling a lock component with the lid of the casing, wherein the lock component is configured to prevent rotational movement of the plunger rod guide relative to the casing and lock the spring in an energized state when the lock component is coupled with the lid.

35. The method of claim 34, wherein the bottom surface, the plurality of sidewalls, and the lid of the casing are integrally formed and stamped from a single sheet of metal.

36. (canceled)

37. The method of claim 34, wherein the lid includes one or more cut-out portions, and the lock component includes at least one hook adjacent to a distal end thereof, and wherein selectively coupling the lock component with the lid of the casing further comprises selectively engaging the at least one hook with the one or more cut-out portions to lock the spring in the energized state.

38. The method of claim 34, wherein the lock component is configured to receive at least a proximal portion of the plunger rod guide when the lock component is coupled with the lid of the casing to prevent rotational movement of the plunger rod guide relative to the casing.

39. (canceled)

40. The method of claim 34, wherein the spring is a torsion spring, and wherein the metal band includes a first hook at a first end thereof and a second hook at a second end thereof, the first hook being configured to engage at least a portion of the plunger rod guide to lock the first end of the metal band onto the plunger rod guide and the second hook being configured to engage a sidewall of the casing.

41. (canceled)

42. The method of claim 34, further comprising:
axially assembling the drive assembly into a drug delivery device while the lock component is coupled with the lid of the casing; and
removing the lock component from the casing.

43. A method of manufacturing a drive assembly for a drug delivery device, the method comprising:
stamping, via a stamping tool, a sheet of metal to form a bottom surface, a plurality of sidewalls, and a lid of a casing, the bottom surface, the plurality of sidewalls, and the lid being integrally formed;
cutting, via a cutting tool, the bottom surface, the plurality of sidewalls, and the lid from the sheet of metal;
folding the bottom surface, the plurality of sidewalls, and the lid to form the casing;
inserting at least a portion of a plunger rod guide through a cavity formed through the bottom surface and the lid of the casing;
feeding a metal band into the casing through an opening in the plurality of sidewalls of the casing;
winding the metal band around a portion of the plunger rod guide within the casing to form a spring; and
selectively coupling a lock component with the lid of the casing, wherein the lock component is configured to prevent rotational movement of the plunger rod guide relative to the casing and lock the spring in an energized state when the lock component is coupled with the lid.

44. The method of claim 43, wherein:
the casing further includes at least one opening formed through the lid,
at least one sidewall of the plurality of sidewalls includes a protrusion extending from the at least one sidewall, and
the method further comprises clinching the protrusion through the at least one opening on the lid to lock the lid in a closed configuration.

45. The method of claim 43, further comprising:
forming a first hook at a first end of the metal band;
forming a second hook at a second end of the metal band;
coupling the first hook to at least a portion of the plunger rod guide to lock the first end of the metal band onto the plunger rod guide; and
coupling the second hook to a sidewall of the casing.

46.-50. (canceled)

51. A drug delivery device, comprising:
a housing having a proximal end, a distal end, and a longitudinal axis extending between the proximal end and the distal end thereof;
a container including a needle and filled or fillable with a drug, the container being disposed within the housing; and
the drive assembly of claim 1, wherein the drive assembly is disposed within the housing and activatable to drive a plunger in a distal direction to expel the drug from the container through the needle.

52.-62. (canceled)

63. The drug delivery device of claim 51, wherein, upon activation of the drive assembly, the plunger rod guide is configured to receive a force output by the spring and rotate relative to the housing, and wherein the plunger is threadingly engaged with the plunger rod guide such that a rotational movement of the plunger rod guide causes the plunger to rotate and translate linearly in the distal direction to expel the drug from the container.

64. (canceled)

65. The drug delivery device of claim 51, wherein the plunger is at least partially disposed within the plunger rod guide.
